(19) United States
(12) Patent Application Publication
(10) Pub. No.: US 2018/0284126 A1
(43) Pub. Date: Oct. 4, 2018

(54) A METHOD OF DETECTING MOLECULES IN PROXIMITY TO A TARGET MOLECULE IN A SAMPLE

(71) Applicant: THE UNITED STATES OF AMERICA AS REPRESENTED BY THE SECRETARY OF THE DPT OF HEALTH AND HUMAN SERVICES, Rockville, MD (US)

(72) Inventors: Daniel Zvi Bar, Rockville, MD (US); Francis Collins, Rockville, MD (US)

(73) Assignee: The United States of America as Represented by the Secretary of the Dpt. of Health and Human Service, Rockville, MD (US)

(21) Appl. No.: 15/756,236

(22) PCT Filed: Aug. 29, 2016
(86) PCT No.: PCT/US2016/049263

§ 371 Date: Feb. 28, 2018

Related U.S. Application Data
(60) Provisional application No. 62/211,160, filed on Aug. 28, 2015.

(51) Int. Cl. G01N 33/68; G01N 33/535

(52) U.S. Cl. CPC G01N 33/6845; G01N 33/535

(57) ABSTRACT

Antibody-based methods of detecting of molecules interacting with a target molecule, such as a nuclear or cytoplasmic protein, in a fixed and, optionally, permeabilized cell or tissue sample. In one example, the target molecule is bound by a primary antibody, and a secondary, peroxidase-conjugated antibody, then binds to the primary antibody. The peroxidase may also be conjugated the primary antibody. Biotin tyramide is added to the sample, and the peroxidase generates short-lived intermediates resulting in biotinylation of molecules adjacent to the peroxidase. Biotinylated molecules can be isolated from the sample by affinity interaction with avidin- or streptavidin, resulting in a fraction containing biotinylated molecules that are located in the sample in proximity to the target molecule. The fraction can be analyzed by mass-spectroscopy, Western blotting, sequencing and other techniques.

### PRIOR RELATED APPLICATION

The present application claims the benefit of priority of U.S. Provisional Application No. 62/211,160 filed Aug. 28, 2015, incorporated herein by reference in its entirety.

### FIELD OF THE INVENTION

The present invention lies in the fields of biochemistry and cell biology and concerns methods and kits useful for identification of molecules in proximity to target molecules in a sample. Some embodiments of the invention relate to detection of protein-protein interactions and/or antibody based labelling. The methods and kits described herein are useful for research, clinical, diagnostic and other applications.

### BACKGROUND

Identification of protein-protein interactions (PPI) is a major challenge in biology. One common method of detecting PPIs is co-immunoprecipitation (CoIP), followed by mass spectrometry. This method suffers from a number of disadvantages. For example, not all the antibodies can immunoprecipitate their antigens, and weak or transient PPIs are difficult to detect by CoIP. Some of the other reasons for poor CoIP results include failure of the antibodies to successfully access their targets (such as nuclear or cytoplasmic proteins) due to sample preparation protocols that attempt to preserve PPI and occurrence of high order structures that lead to isolation of multiple unrelated proteins and mask the signal from the PPIs. Another class of methods for detecting PPI is proximity labeling. Proximity labelling methods rely on deposition of a tag, usually biotin, on the proteins adjacent to the target of interest. A major limitation of current implementations of proximity labeling is the need to generate new transgenic cell lines and animals for every protein of interest probed. Additionally, currently available proximity labeling protocols are not necessarily applicable to human primary tissue samples.

### SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are further described in the Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

Described herein and included among the embodiments of the present invention are methods of detecting molecules in proximity to a target molecule in a sample. The sample used for detection in some embodiments of the methods of the present invention is a fixed and, optionally, permeabilized cell or tissue sample. The molecules that can be detected according to the methods of the present invention may be referred to as “analytes” or “analyte molecules,” and include biological as well as non-biological molecules. The target molecules also include biological as well as non-biological molecules. The biological molecule in the context of the methods of the present invention may be a protein, such as a nuclear or a cytoplasmic protein, or an extracellular/secreted protein, or another type of biological molecule or a part thereof, including, but not limited to, a lipid, a nucleic acid, a fatty acids or an oligosaccharide. The non-biological molecule may be a small molecule, such as a drug.

The methods described herein have a number of advantages over previously known methods of detecting molecular interactions in biological samples, such as CoIP or previously known proximity labelling methods. At least some of these advantages are unexpected and are discussed further in this document. In the embodiments of the methods described herein, a target molecule found in a fixed and, optionally, permeabilized cell or tissue sample is bound by a primary antibody or a molecule that is specific for the target molecule. In some embodiments, a secondary, peroxidase-conjugated antibody, then binds to the primary antibody or other primary binding molecule. In some other embodiments, the secondary antibody is not employed. Instead, the peroxidase is conjugated or otherwise included in the primary antibody or primary binding molecule. A non-limiting example of a peroxidase is horseradish peroxidase (HRP). Biotin tyramide, also known as biotin phenol, is added to the sample as a substrate for the peroxidase. In the presence of hydrogen peroxide, short-lived intermediates are generated by the peroxidase, resulting in biotinylation of suitable moieties on the adjacent molecules. For example, Tyr, Trp, His and Cys residues on the proteins adjacent to the primary antibody or other binding molecule may be biotinylated. Nucleic acids can also be biotinylated, as well as other molecules.

The molecules that are biotinylated using the above process and its variations, which can be referred to as “proximity biotinylation,” “proximity labelling,” “tyramide labeling,” “antibody-based labeling” or by other related terms, can be analyzed by various techniques. For example, visualization of biotinylated residues in the sample can be performed by fluorophore-bound avidin, which binds biotin. The molecules containing biotinylated moieties, such as the proteins containing biotinylated amino acids, can be isolated from the sample. The proteins in the sample can be subjected to solubilization, for example, by elevated temperature and/or other treatments, to reverse of the effects of fixation (for example, reversal of formaldehyde cross-linking) aimed at disrupting protein-protein interactions in the sample and making the proteins and other molecules in the sample accessible for analysis by mass-spectrometry and other methods. After the sample is treated in such a manner, the molecules containing biotinylated moieties, such as biotinylated proteins and polypeptides, can be isolated by affinity interaction with avidin- or streptavidin, for example, by using avidin or streptavidin-coated beads. An affinity-based purification procedure results in isolation of a fraction containing biotinylated molecules or parts of such molecules (for example, biotinylated proteins or polypeptides, biotinylated nucleic acids, etc.) that were located in the sample in proximity to the target molecule. The fraction can then be analyzed by mass-spectrometry, Western blotting, sequencing and other techniques, to identify the molecules that were located in the sample in proximity to the target molecule.

In an illustrative example of the methods described herein, an embodiment of the method of the present invention was used to analyze and identify the proteins and polypeptides in proximity to lamin A/C in various cells and tissue samples fixed with formaldehyde. A commercially available monoclonal anti-lamin A/C antibody MAB3211 (Merck Millipore) was employed. The samples, including cell lines and human primary tissues, were solubilized and formaldehyde cross-linking was reversed prior to the affinity purification step. Affinity purification was performed using streptavidin-coated beads. The isolated fraction was analyzed by mass spectrometry and Western blotting. In some cases, ratiometric calculations of the bound over unbound to streptavidin-coated beads fractions were employed to distinguish lamin-interacting proteins from background contamination by abundant proteins. In some cases, stable isotope labeling by amino acids in cell culture (SILAC) was used to detect changes in interacting proteins under specific conditions, such as heat shock or expression of a mutant form of lamin A (progerin). The above illustrative example may be better understood in reference to FIG. 1.

Some embodiments of the present invention are summarized below. One exemplary embodiment is a method of identifying one or more analyte molecules in proximity to a target molecule in a sample, comprising: contacting the sample with a primary binding molecule under primary molecule binding conditions, which allow binding of the target molecule in the sample to the primary binding molecule to occur, wherein the sample is a fixed and optionally permeabilized sample of a cell or a tissue; subsequent to contacting, exposing the sample to a secondary antibody under secondary antibody binding conditions allowing binding of the secondary antibody to the primary binding molecule to occur in the sample, wherein the secondary antibody comprises a peroxidase; subsequent to exposing, adding biotin tyramide to the sample under biotinylation conditions allowing for biotinylation of one or more moieties of the one or more analyte molecules located in proximity to the peroxidase, thereby resulting in one or more biotinylated moieties; subsequent to adding, treating the sample under conditions allowing for protein solubilization; subsequent to treating, isolating from the sample the one or more analyte molecules or one or more parts thereof comprising the one or more biotinylated moieties, by an isolation process comprising affinity interaction of the one or more biotinylated moieties and an affinity reagent; and, analyzing the one or more analyte molecules or the one or more parts thereof. In the above exemplary embodiment, a secondary antibody can be conjugated to the peroxidase. Another exemplary embodiment is a method of identifying one or more analyte molecules in proximity to a target molecule in a sample, comprising: contacting the sample with a primary binding molecule under primary molecule binding conditions, which allow binding of the target molecule in the sample to the primary binding molecule to occur, wherein the sample is a fixed and optionally permeabilized sample of a cell or a tissue, and wherein the primary binding molecule comprises a peroxidase; subsequent to contacting, adding biotin tyramide to the sample under biotinylation conditions allowing for biotinylation of one or more moieties of the one or more analyte molecules located in proximity to the peroxidase, thereby resulting in one or more biotinylated moieties; subsequent to adding, treating the sample under conditions allowing for protein solubilization; subsequent to treating, isolating from the sample the one or more analyte molecule or one or more parts thereof comprising the one or more biotinylated moieties, by an isolation process comprising affinity interaction of the one or more biotinylated moieties and an affinity reagent; and, analyzing the one or more analyte molecules or the one or more parts thereof. In the above exemplary embodiment, the primary binding molecule may be conjugated to the peroxidase.

In the embodiments of the methods described herein, the target molecule may be a biological or a non-biological molecule. For example, the target molecule may be a protein, a polypeptide, a lipid, a fatty acids, an oligosaccharides, a nucleic acids, or a small molecule. The small molecule may be a drug or a receptor ligand. The target molecule may be a protein or a polypeptide, in which case one or more biotinylated moieties are one or more amino acids. In the embodiments of the methods described herein, in the analyzing step, the proteins may be analyzed by Western blot, high throughput protein identification methods, including mass spectrometry. In the embodiments of the methods described herein, the analyte molecule may be a biological or a non-biological molecule. The analyte molecule may be a protein, a polypeptide, a lipid, a fatty acid, an oligosaccharide, a nucleic acid, or a small molecule. The small molecule may be a drug. The analyte molecule is a protein or a polypeptide. In the embodiments of the methods described herein, the primary binding molecule may be a primary antibody. In one example, the primary antibody is an anti-lamin A/C antibody.

In the embodiments of the methods described herein, the fixed sample may be a sample fixed by a reversible cross-linking reagent. The reversible cross-linking reagent may formaldehyde or acrolein. When a cross-linking reagent is employed, a treating step may comprise exposing the sample to cross-linking reversal conditions allowing for at least partial reversal of the effects of the reversible cross-linking reagent. Such cross-linking reversal conditions may include one or both of: incubation of the sample at a temperature of 55-99° C. and incubation of the sample in the presence of NaCl. In the embodiments of the methods described herein, the fixed sample may be a sample fixed by a precipitating fixative. The precipitating fixative may comprise one or more of ethanol, methanol or acetone. In the embodiments of the methods described herein, wherein the treating step comprises exposing the sample to a detergent, an elevated temperature, or both, under conditions allowing at least partial solubilization of proteins in the sample. In some embodiments of the present invention, the sample is permeabilized. The sample may be permeabilized by exposure to a detergent. In some examples, the detergent comprises one or more of Triton X-100, Polyoxyethylene (20), sorbitan monooleate (Tween-20) or saponin. In the embodiments of the methods described herein, the sample may be a tissue sample having a thickness of 0.1-5 mm. In the exemplary embodiments of the methods described herein, the affinity interaction is biotin-avidin or biotin-streptavidin interaction. The affinity reagent may be a plurality of beads coated with avidin or streptavidin.

Some additional examples of the embodiments of the present invention are as follows. The method, in which the target molecule is a mutant protein, and the primary binding molecule is a primary antibody specific for the mutant protein. The method, in which the target protein is a modified protein, and the primary binding molecule is a primary antibody specific for the mutant protein. The method, in which the target molecule is a lipid or a fatty acid, and the primary binding molecule is a primary antibody specific for the lipid or the fatty acid. The method, in which the target molecule is an oligosaccharide, and the primary binding molecule is a primary antibody specific for the oligosaccharide. The method, in which the target molecule is a first nucleic acid, and the primary binding molecule is a second nucleic acid capable of specifically binding the first nucleic acid. The method, in which the analyte molecule is or comprises a lipid, a fatty acid, a nucleic acid or an oligosaccharide, and in the analyzing step mass spectrometry is used to identify the lipid, the fatty acid, the nucleic acid or the oligosaccharide. The method, in which analyte molecule is or comprises a nucleic acid, and in the analyzing step sequencing is used to identify the nucleic acid. The method, in which the target molecule is a drug, a biological or a small molecule, and the primary binding molecule is a primary antibody specific for the drug, the biological or the small molecule. The method, in which the target molecule is a receptor or ligand, and the primary binding molecule is a primary antibody specific for the receptor or the ligand. The method, in which the target molecule is a modified protein having a posttranslational modification, and the primary binding molecule is a primary antibody specific for the protein having the posttranslational modification. The posttranslational modification may be one or more of glycosylation, acetylation, phosphorylation or methylation. The method, in which the target molecule is a protein isoform, and wherein the primary binding reagent is a primary antibody specific for the protein isoform. The method, in which the target molecule is a nuclear or a cytoplasmic protein. The method, in which the target molecule is a nuclear envelope protein. The method, in which the target molecule is a lamin A/C. The method, in which the target molecule is a histone. The method, in which the target molecule is a protein selected from the group consisting of CTCF, P53, P53BPI, ATM, CHK1, CHK2, ATR, PRKDC, CAK, TP53RK, RAS, an mTOR complex protein, WRN, Ku70, Ku80, SREBP, APOE, LAP2 and a NF-κB complex protein.

One more exemplary embodiment of the present invention is a method of identifying one or more analyte molecules in proximity to a target molecule in a sample of a cell or a tissue, comprising: contacting the cell or the tissue with the target molecule under incorporation conditions allowing of incorporation of the target molecule into the cell or the tissue, wherein the target molecule comprises a peroxidase; after the incorporation, preparing the sample of the cell or the tissue, wherein the sample is a fixed and optionally permeabilized sample of a cell or a tissue, and; adding biotin tyramide to the sample under biotinylation conditions allowing for biotinylation of one or more moieties of the one or more analyte molecules located in proximity to the target molecule, thereby resulting in one or more biotinylated moieties; subsequent to adding, treating the sample under conditions allowing for protein solubilization; subsequent to treating, isolating from the sample the one or more analyte molecules or one or more parts thereof comprising the one or more biotinylated moieties, by an isolation process comprising affinity interaction of the one or more biotinylated moieties and an affinity reagent; and analyzing the one or more analyte molecules or the one or more parts thereof. The peroxidase may be conjugated to the target molecule, for example, by click chemistry. In some exemplary variations of the above embodiment, the target molecule is a protein, a peptide, a small molecule, a drug, a nucleic acid, or a ligand.

Other embodiments, objects and advantages of the invention will be apparent from the following detailed description of embodiments of the invention.

### BRIEF DESCRIPTION OF THE FIGURES

FIG. 1 is a schematic illustration of an embodiment of a method of the present invention.

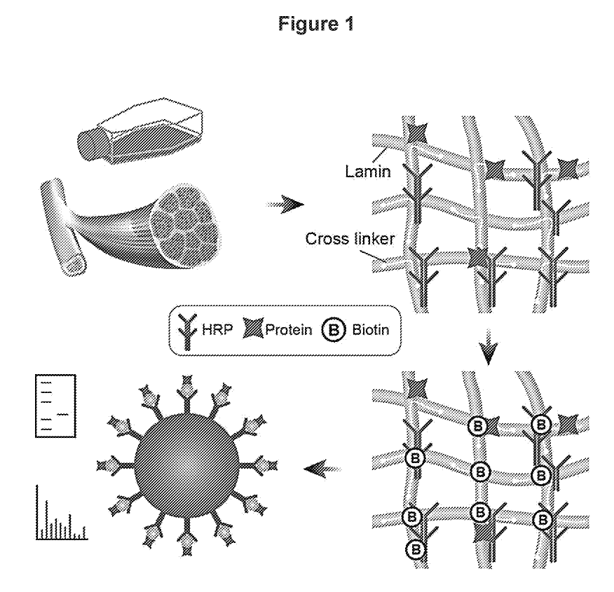

FIG. 2 shows the images illustrating the results obtained by the embodiments of a method of the present invention. Top left panel “HeLa” shows an image of super-resolution microscopy showing biotin deposition in the vicinity of the nuclear envelope in HeLa cells, confirming the tight spatial resolution of the protein labeling method (Lamin A—labelling with a fluorescent dye-conjugated secondary antibody; Biotin—labelling with FITC-avidin). The image was acquired with a ZEISS LSM 880 with Airyscan system equipped with a Plan-Apochromat 63×/1.40 Oil DIC M27 objective and using 488 and 561 nm wavelength lasers. Top right panel “Adipose” shows the images illustrating identification of nuclear envelope proteins in primary human adipose tissue; doughnut shaped nuclei from primary human adipose tissue are visible (Lamin—labelling with a fluorescent Cy3-conjugated secondary antibody; Biotin—labelling with FITC-avidin; DNA—labeling with DAPI: Merge—superimposed of the previous three images). Bottom panel “Muscle” shows the images illustrating the imaging of primary human skeletal and smooth muscle tissue (Lamin A/C—labelling with a fluorescent Cy3-conjugated secondary antibody; Biotin—labelling with FITC-avidin; DNA—DAPI). The images in “Adipose” and “Muscle” panels were acquired with a DeltaVision PersonalDV (Applied Precision) with either a Plan Apo 60×/1.42 NA or a UPlanSApo 100×/1.4 NA oil lenses

FIG. 3 shows an image of a Western blot of the proteins isolated by affinity interaction with streptavidin beads from the samples subjected to proximity labelling using anti-lamin primary antibody (“L”) and negative control samples subjected to the same procedures in the absence of the primary antibody (“C”). The left lane contains molecular weight controls. The approximate location of lamin interactor LAP2B band is indicated (“LAP2B”).

FIG. 4 shows, in panel A, an image of a Western blot showing antibody-guided efficient biotin labeling and recovery of proteins isolated by affinity interaction with streptavidin beads from the samples subjected to proximity labelling using anti-lamin primary antibody (“+”) and negative control samples subjected to the same procedures in the absence of the primary antibody (“−”). The left lane contains molecular weight controls. Panel B is an image of a Western blot illustrating isolation of LAP2, a known lamin A/C binding protein from the samples subjected to proximity labelling using anti-lamin primary antibody (“+”) and negative control samples subjected to the same procedures in the absence of the primary antibody (“−”). The left lane contains molecular weight controls. The approximate location of lamin interactor LAP2a and LAP2B bands is indicated.

FIG. 5 is an image of a screenshot of the “scaffold” generated by Proteome Software, Portland, Oreg., showing multiple known nuclear envelope proteins and lamin A/C interacting proteins, and their peptide counts in the antibody (+) and control (−) samples obtained during the experiments illustrated in FIG. 4, panel B..

FIG. 6 shows a dot plot illustrating bound over unbound enrichment score discriminating known nuclear envelope proteins from abundant ribosomal proteins. Enrichment score for ribosomal proteins and various nuclear pore proteins (as indicated) is plotted on the Y axis.

FIG. 7 illustrates enriched of GO terms in ranked lists of genes of the proteins isolated after a proximity-labelling experiment of HeLa cells samples using anti-lamin A/C primary antibody. Color coding indicates p-value, calculated using “Gene Ontology enRIchment anaLysis and visuaLizAtion tool” for identifying and visualizing enriched GO terms in ranked lists of genes (Eden et al. “GOrilla: A Tool For Discovery And Visualization of Enriched GO Terms in Ranked Gene Lists,” BMC Bioinformatics 2009, 10:48.; Eden et al. “Discovering Motifs in Ranked Lists of DNA sequences”, PLoS Computational Biology, 3(3):e39, 2007).

FIG. 8 is a bar graph illustrating comparison of three published datasets of laming-binding proteins and the dataset obtained using the methods of the present invention (as indicated). Each bar in the graph represents a calculated average percentage of the other datasets covered by the indicated dataset.

FIG. 9 shows a dot plot illustrating a correlation between nuclear envelope proteins obtained from HeLa cells grown in heavy and light SILAC medium. Dot size indicates with the number of peptides detected.

FIG. 10 illustrates the results of a proof of concept study of HeLa cells using proximity labelling in conjunction with SILAC. HeLa cells were subjected to 43° C. heat shock for 2 hours. Heat-shock induced changes to the composition of the nuclear lamina were evaluated. Control HeLa cells were not subjected to the heat shock. Upper panel shows the peptide profile illustrating comparison of “heavy” vs. “light” control cells. Lower panel shows peptide profiles of the “heavy” heat shocked cells vs. “light” control cells (blue) and “light” heat shocked cells vs “heavy” control cells (X-axis shows the number of peptides of the blue label used to calculate the fold change).

FIG. 11 illustrates the results of the proximity labelling of transfected HeLa cells. Panel A shows an image Ku70 subcellular localization in HeLa cells, visualized with immunofluorescence, before (Control) and after heat shock (HS). Scale bar—10 m. The images were acquired on a Leica TCS SP5 confocal microscope equipped with a HCX PL APO CS 63.0×/1.40 NA oil lens

FIG. 12 shows an image obtained by structured illumination super-resolution microscopy of HeLa cells transfected with GFP-LMNA or GFP-Progerin. The images were acquired with a OMX Structured Illumination Super-resolution Scope equipped with a PlanApo 60×/1.40 Oil DIC objective and using 488 and 568 nm wavelength lasers.

FIG. 13 shows a dot plot illustrating “Heavy (lamin A/C) to light (progerin) peptide ratio of GFP-LMNA/Progerin in transfected HeLa cells. Progerin protein model shown under the X-axis indicates the location of the 50 amino-acid deletion, overlapping with the last 3 peptides.

FIG. 14 is a dot plot illustrating Heavy (lamin A/C) to light (progerin) DNA-PKcs peptide ratio in GFP-LMNA/Progerin transfected HeLa cells.

FIG. 15 shows an image obtained by structured illumination super-resolution microscopy of control and HGPS-derived fibroblasts. Scale bar—1 μm. The images were acquired with a OMX Structured Illumination Super-resolution Scope equipped with a PlanApo 60×/1.40 Oil DIC objective and using 488 and 568 nm wavelength lasers.

FIG. 16 is a dot plot illustrating Nesprin-1 peptide ratio in control “heavy” vs HGPS “light” fibroblasts.

FIG. 17 shows the images illustrating CAV1 immunofluorescence in primary human muscle tissue. Scale bar—10 μm

### DESCRIPTION

Embodiments of the present invention provide improved methods of detecting proteins and other molecules interacting with or in proximity to a target molecule in a fixed and, optionally, permeabilized cell or tissue sample. Such methods may be referred to as “detection methods.” The kits for performing such detections methods, as well as the methods embodying the analytical, diagnostic clinical and therapeutic applications of the detection methods are also included among the embodiments of the present invention. The definitions and explanations of certain terms and expressions that may be employed to describe the embodiments of the present invention are provided to facilitate the understanding of the invention. The definitions are not intended to be exhaustive. These definitions and explanations may be found below and elsewhere in this documents.

### Definitions

As used herein, the terms “a,” “an,” and “the” can refer to “one,” “one or more” or “at least one,” unless specifically noted otherwise.

The term “about” is used herein to indicate that a value includes the inherent variation of error for the device, the method being employed to determine the value, or the variation that exists among the study subjects.

As used herein, the term “biomarker” or “biomarker molecule” is used to denote any molecule or a combination of molecules that may provide biological information about the physiological state of an organism. In some cases, the presence or absence of the biomarker may be informative. In some other cases, the level of the biomarker may be informative.

The term “occurrence” may be used herein to denote an incidence of molecules, for example, proteins, as well as frequency of their appearance, quantity, or distribution throughout different classes or subclasses. In some embodiments of the present invention, any of the foregoing information falling within the meaning of the term “occurrence” can be utilized in relation to one or more proteins, as well as classes and subclasses of such proteins. The combination of such information can be referred to as a “pattern.” The information on occurrence of molecules or patterns, such as the information obtained in the course of performing the methods described herein can be compared or correlated with the information previously obtained, processed or stored. The results of such comparison, according to certain embodiments of the present invention, can lead to assessment or detection of a disease or a condition. When the information on occurrence of molecules or patterns is derived from a sample obtained from a human or an animal patient or subject, the method is useful for detection of a disease or a condition in the subject, or for determining a predisposition to develop a disease or condition at some future time. In other words, the occurrence of molecules or patterns using the methods described herein can be used in the present invention as a characteristic measured and evaluated as an indicator of certain biological processes, diseases or conditions.

The terms “assess,” “assessment” and similar terms are used herein to broadly refer to a process of discovering or determining the presence or an absence, as well as a degree, quantity, or level, or probability of occurrence of something. For example, the term “assess” and related terms, when used in reference to a molecule, such as a protein or a peptide, denote discovery or determination of the presence of the molecule, absence of the molecule, quantity, level or estimated quantity or level of the molecule, changes in the quantity or level of the molecule (for example, over time or under different conditions) as well as a probability of presence or absence of the molecule. The term “assess” and related terms can be used interchangeably with the term “monitor,” “detect,” “detecting,” “indicate,” and other related terms. When used in reference to a disease or condition, the term “assess” and related terms can denote discovery or determination of presence of a disease or a condition, absence of a disease or a condition, progression, level or severity of a disease or a condition, as well as a probability of present or future exacerbation of symptoms, or of efficacy of a treatment. For example, evaluating prognosis of a disease or condition in a subject falls within the scope of the terms “assess,” “assessment,” “assessing” and the related terms. The terms “assess,” “assessment,” “assessing” and related terms may also encompass, depending on the context, recommending or performing additional analytical or diagnostic procedures, evaluating potential effectiveness of the treatments or therapies, as well as recommending or performing such treatments or therapies, monitoring a medical disease or conditions, or any other steps or processes related to treatment or diagnosis of a disease or a condition. These terms also encompass recommending or not recommending and performing or not performing treatment or diagnostic procedures, as well as recommending or not recommending and performing or not performing palliative or hospice care.

The terms “analysis” or “analyzing” and similar terms are used herein to broadly refer to studying or determining or identifying a nature, properties, or quantity of an object under analysis, or its components. Analysis can include assessment or detection, as discussed above. Analysis can include studying, determining or identifying changes, for example, changes over time or under different conditions. Analysis can also involve chemical or biochemical manipulations or steps, as well as manipulations or steps of nature, as well as manipulation of information in an appropriate manner (for example, storage of information in computer memory and computer calculations may be used). The terms “analysis” and the related terms can be used interchangeably with the terms “assessment,” “detection,” “identification,” “monitoring” and other related terms.

As used herein, the terms “subject,” “individual,” and “patient” are used interchangeably. The use of these terms does not imply any kind of relationship to a medical professional, such as a physician or a veterinarian. The term “subject” and related terms refer to an organism. Subject may be a mammal such as a primate, including a human. The term “subject” includes non-human animals, for example, domesticated animals, such as cats, dogs, etc., livestock (e.g., cattle, horses, pigs, sheep, goats, etc.), and laboratory animals (e.g., mouse, rabbit, rat, guinea pig, etc.). The term “subject” as used may refer to a subject, such as, but not limited to, a human person, having a medical disease or condition, such as a laminopathy. It is to be understood, that a subject having a medical disease or condition can be a patient with a known disease or condition, meaning the disease or condition that was detected prior to the performance of the embodiments of the methods of the present invention, or a subject with a previously undetected disease or condition.

The term “condition” when used in reference to the embodiments of the invention disclosed herein is used broadly to denote a biological state or process, such as an immune or inflammatory response, which can be normal or abnormal or pathological. The term “condition” can be used to refer to a medical or a clinical condition, meaning broadly a process occurring in a body or an organism and distinguished by certain symptoms and signs. The term “condition” can be used to refer to a disease or pathology, meaning broadly an abnormal disease or condition affecting a body or an organism.

Some examples of conditions that can be usefully assessed using the methods described herein are nuclear envelopathies, including laminopathies, meaning broadly a group of disorders caused by variations in the proteins of the nuclear envelope, such as, in the case of laminopathies, nuclear lamina. Nuclear envelopathies can have diverse clinical phenotypes, such as, but not limited to, cardiac and skeletal myopathies, lipodystrophy, peripheral neuropathy, diabetes, dysplasia, dermopathy, neuropathy, leukodystrophy and premature aging. Nuclear envelopathies may be caused by mutations in a gene or genes coding for lamin-binding proteins such as lamin B receptor (LBR gene), emerin (EMD gene), LEM domain-containing protein 3 (LEMD3 gene), prelamin-processing enzymes such as the zinc metalloproteinase STE24 (ZMPSTE24 gene). Examples of laminopathies are laminopathies caused by mutations in the gene coding for lamin A/C (LMNA gene) or lamin B (LMNB2 gene). Some types of premature aging, such as progeria, in particular, Hutchinson-Gilford progeria syndrome, may be classified as laminopathies or nuclear envelopathies.

The term “nuclear envelope” is used herein to refer to outer structures of a nucleus of an eukaryotic cells, encompassing a nuclear membrane (including an inner and outer membrane), nuclear membrane associated proteins, nuclear lamina and nuclear pores The term “nuclear lamina” is used to describe a filamentous network outside of nucleus composed of intermediate filaments and associated proteins.

The term “lamin” is used herein to refer to lamin proteins and polypeptides providing structural function and transcriptional regulation in the cell nucleus, including B-type lamins, such as those enclosed by human LMNB1 and LMNB2 genes, and A-type lamins, such as lamins A and C encoded, as splice variants, by the LMNA gene.

The terms “sample” or “samples” as used herein are not intended to be limiting unless qualified otherwise and refer to any product, composition, cell, tissue or organism that may contain targets, such as target proteins, in the context of the methods described in this document. For example, “sample” may be any cell or tissue sample or extract originating from cells, tissues or subjects, and include samples of human or animal cells or tissues as well as cells of non-human or non-animal origin, including bacterial samples. A sample can be directly obtained from a human or animal organism, or propagated or cultured. Samples can be subject to various treatments, storage or processing procedures before being analyzed according to the methods described in the document. Generally, the terms “sample” or “samples” are not intended to be limited by their source, origin, manner of procurement, treatment, processing, storage or analysis, or any modification. Samples include, but are not limited to, samples of human cells and tissues. Samples encompass samples of healthy or pathological cells, tissues or structures. Samples can contain or be predominantly composed of cells or tissues, or can be prepared from cells or tissues. Some examples of the samples are solutions, suspensions, supernatants, precipitates (cell precipitates), pellets, cell extracts (for examples, cell lysates), cell extracts, blood or plasma samples, tissue sections and/or including needle biopsies, microscopy slides, including fixed tissues (ex. formalin-fixed, paraffin-embedded (FFPE)) or frozen tissue sections, flow cytometry samples and fixed cell and tissue samples.

As used herein, the term “specific binding molecule” is used to denote a molecule capable of specifically or selectively binding another molecule or a region or structure within another molecule, which may be termed “target,” “ligand” or “binding partner.” As used herein, the terms “specific binding,” “selective binding” or related terms refer to a binding reaction in which, under designated conditions, a specific binding molecule or a composition containing it binds to its binding partner or partners and does not bind in a significant amount to anything else. Binding to anything else other than the binding partner is typically referred to as “nonspecific binding” or “background.” The absence of binding in a significant amount is considered, for example, to be binding less than 1.5 times background (i.e., the level of non-specific binding or slightly above non-specific binding levels). Some nonlimiting examples of specific binding are antibody-antigen or antibody-epitope binding, binding of oligo- or polynucleotides to other oligo- or polynucleotides, binding of oligo- or polynucleotides to proteins or polypeptides (and vice versa), binding or proteins to polypeptides other proteins or polypeptides or receptor-ligand binding. Accordingly, specific binding molecules can be or can include a protein, a polypeptide, an antibody, an oligo- or polynucleotide, a receptor, or a ligand. This list is not intended to be limiting, and other types of specific binding molecules may be employed. The term “primary binding molecule” is used herein to denote a specific binding molecule capable of specifically binding a target molecule in the context of the methods according to the embodiments of the present invention. A primary binding molecule can be an antibody, which can be referred to as a “primary antibody.” In contrast, “secondary binding molecule” is a molecule capable of specifically binding a “primary binding molecule,” such as primary antibody, but not the target molecule.

The term “target molecule” is used herein to detect a molecule or a part thereof, including a biological molecule (such as, but not limited to, a protein, a peptide, lipid, a nucleic acid, a fatty acid, or a carbohydrate molecule, such as an oligosaccharide), or a nonbiological molecule (including a small molecule, such a small molecule drug or a small molecule ligand). A primary specific binding molecule, such as a primary antibody, specifically binds to the target molecule when the methods of the present invention are performed.

The term “analyte molecule” is used herein to refer to a molecule or a part thereof interacting with or in proximity to the target molecule. Analyte molecule is detected by the methods according to the embodiments of the present invention. Analyte molecules include biological molecules (such as, but not limited to, proteins, peptides, lipids, nucleic acids, fatty acids or carbohydrates, such as oligosaccharides) and nonbiological molecules (including small molecules, such a small molecule drugs or small molecule ligands).

The term “antibody” and the related terms, in the broadest sense, are used herein to denote any product, composition or molecule that contains at least one epitope binding site, meaning a molecule capable of specifically binding an “epitope”—a region or structure within an antigen. The term “antibody” encompasses whole immunoglobulin (i.e., an intact antibody) of any class, including natural, natural-based, modified and non-natural antibodies, as well as their fragments. The term “antibody” encompasses “polyclonal antibodies,” which react against the same antigen, but may bind to different epitopes within the antigen, as well as “monoclonal antibodies” (“mAbs”), meaning a substantially homogenous population of antibodies or an antibody obtained from a substantially homogeneous population of antibodies. The antigen binding sites of the individual antibodies comprising the population of mAbs are comprised of polypeptide regions similar (although not necessarily identical) in sequence. The term “antibody” also encompasses fragments, variants, modified and engineered antibodies, such as those artificially produced (“engineered), for example, by recombinant techniques. For instance, the term “antibody” encompasses, but is not limited to, chimeric antibodies and hybrid antibodies, antibodies with dual or multiple antigen or epitope specificities, and fragments, such as F(ab′)2, Fab′, Fab, hybrid fragment, single chain variable fragments (scFv), “third generation” (3G) fragments, fusion proteins, single domain and “miniaturized” antibody molecules.

The term “peroxidase” is used herein to denote an enzyme that typically catalyzes a reaction of the form: ROOR′+electron donor (2 e⁻)+2H⁺—ROH+R′OH. A peroxidase that can be used in the methods described herein is capable of using a biotin tyramide compound, also known as biotin phenol, as a substrate, and converting it to a highly reactive free radical that binds covalently to electron-rich amino acids, resulting in their biotinylation. The chemical principles of tyramide reaction and its applications in protein labelling methods are described in U.S. Pat. No. 5,731,158 and McKay et al., “Amplification of fluorescent in situ hybridization signals in formalin fixed paraffin wax embedded sections of colon tumour using biotinylated tyramide,” J. Clin. Pathol: Mol. Pathol. 50:322-25, 1997. A peroxidase that can be used in the methods described herein can be a naturally occurring, modified, synthetic or engineered peroxidase. Such a peroxidase may be attached to, conjugated to or otherwise integrated into another molecule. For example, antibodies in the methods described herein may be conjugated to a peroxidase or include a peroxidase active site. The peroxidase active site can be attached to an antibody directly (for example, by being integrated into a polypeptide sequence or chemically conjugated to the polypeptide) or indirectly, by way of binding to a primary antibody or a secondary antibody.

As used herein, the terms “isolate,” “separate” or “purify” or similar terms are not used necessarily to refer to the removal of all materials other than the components of interest from a sample. Instead, in some embodiments, the terms are used to refer to a procedure that enriches the amount of one or more components of interest relative to one or more other components present in the sample. In some embodiments, “isolation,” “separation” or “purification” may be used to remove or decrease the amount of one or more components from a sample that could interfere with the detection of the component of interest.

As used herein, the term “mass spectrometry” (MS) refers to a technique for the identification and/or quantitation of molecules in a sample. MS includes ionizing the molecules in a sample, forming charged molecules; separating the charged molecules according to their mass-to-charge ratio; and detecting the charged molecules. MS allows for both the qualitative and quantitative detection of molecules in a sample. The molecules may be ionized and detected by any suitable means known to one of skill in the art. Some examples of mass spectrometry are “tandem mass spectrometry” or “MS/MS,” which are the techniques wherein multiple rounds of mass spectrometry occur, either simultaneously using more than one mass analyzer or sequentially using a single mass analyzer. The term “mass spectrometry” may refer to the application of mass spectrometry to protein analysis. Electrospray ionization (ESI) and matrix-assisted laser desorption/ionization (MALDI) may be used in this context. Intact protein molecules may be ionized by the above techniques, and then introduced to a mass analyzer. Alternatively, protein molecules may be broken down into smaller peptides, for example, by enzymatic digestion by a protease, such as trypsin. Subsequently, the peptides are introduced into the mass spectrometer and identified by peptide mass fingerprinting or tandem mass spectrometry.

As used herein, the term “mass spectrometer” is used to refer an apparatus for performing mass spectrometry that includes a component for ionizing molecules and detecting charged molecules. Various types of mass spectrometers may be employed in the methods of the present invention. For example, whole protein mass spectroscopy analysis may be conducted using time-of-flight (TOF) or Fourier transform ion cyclotron resonance (FT-ICR) instruments. For peptide mass analysis, MALDI time-of-flight instruments may be employed, as they permit the acquisition of peptide mass fingerprints (PMFs) at high pace. Multiple stage quadrupole-time-of-flight and the quadrupole ion trap instruments can also be used.

The terms “high throughput protein identification,” “proteomics” and other related terms are used herein to refer to the processes of identification of a large number (in some cases, all) proteins in a certain protein complement. Post-translational protein modifications and quantitative information can also be assessed by such methods. One example of “high throughput protein identification” is a gel-based process that includes the pre-fractionation and purification of proteins by one-dimensional protein gel electrophoresis. The gel can then be fractionated into several molecular weight fractions to reduce sample complexity, and proteins can be in-gel digested with trypsin. The tryptic peptides are extracted from the gel, further fractionated by liquid chromatography and analyzed by mass spectrometry. In another approach, a sample can be fractionated without using the gels, for example, by protein extraction followed by liquid chromatography. The proteins can then be digested in-solution, and the proteolytic fragments further fractionated by liquid chromatography and analyzed by mass spectrometry

As used herein, the term “Western blot,” which can be also referred to as “immunoblot” and the related terms refer to an analytical technique used to detect specific proteins in a sample. The technique uses gel electrophoresis to separate the proteins, which are then transferred from the gel to a membrane (typically nitrocellulose or PVDF) and stained, in membrane, with antibodies specific to the target protein.

The expression “stable isotope labeling by amino acids in cell culture” (SILAC) is used herein to refer to an approach for in vivo incorporation of a label into proteins for mass spectrometry (MS)-based quantitative proteomics. SILAC relies on metabolic incorporation of a given “light” or “heavy” form of the amino acid into the proteins. SILAC relies on the incorporation of amino acids with substituted stable isotopic nuclei (e.g. deuterium, ¹³C, ¹⁵N). In an illustrative SILAC experiment, two cell populations are grown in culture media that are identical, except that one of them contains a “light” and the other a “heavy” form of a particular amino acid (for example, ¹²C and ¹³C labeled L-lysine, respectively). When the labeled analog of an amino acid is supplied to cells in culture instead of the natural amino acid, it is incorporated into all newly synthesized proteins. After a number of cell divisions, each instance of the amino acid is replaced by its isotope-labeled analog. Since there is little chemical difference between the labeled amino acid and the natural amino acid isotopes, the cells behave substantially similar to the control cell population grown in the presence of a normal amino acid.

The expression “nucleic acid detection methods” is used to refer to the methods, procedures and protocols, using which the nucleic acid sequences are detected. Nucleic acid detection methods can include amplification (such as polymerase chain reaction (PCR)), quantitative amplification (such as quantitative PCR (qPCR)) or nucleic acid sequencing. Amplifications may be monitored in “real time.” Though standard Sanger dideoxy or other older nucleotide sequencing methods can be used, sequencing can be particularly effective when high throughput sequencing is used, e.g., “next generation sequencing” methods such as HiSeq™, MiSeq™, or Genome Analyzer (each available from Illumina), SOLiD™ or Ion Torrent™ (each available from Life Technologies) and 454™ sequencing (from Roche Diagnostics). For example, in high-throughput sequencing, parallel sequencing reactions may be used using multiple templates and multiple primers allows rapid sequencing of genomes or large portions of genomes. Amplicons may be sequenced in a base-incorporation method, a pyrosequencing method, a hydrogen ion detection method, or a dye-terminator detection method. Deep sequencing technology and instruments (i.e., technology and instrument capable of digital sequence readout) may also be employed.

The term “variant” as used herein in reference to proteins encompasses various variants and isoforms, such as splice variants and isoforms, posttranslationally modified variants and isoforms, as well as mutants and homologs. Variants may result from sequence variations, such as amino acid substitutions, deletions, and insertions, as well as from splicing and post-translational modifications. The term “posttranslational modification” and related terms are used herein to refer to the covalent modification of proteins or polypeptide during or after protein biosynthesis. Posttranslational modifications include, but are not limited to, lipidation, acylation, glycosylation, hydroxylation, phosphorylation and methylation. Posttranslational lipidation includes prenylation, such as the attachment of a farnesyl moiety (farnesylation). Variants may arise as a result of the differences in glycosylation, such as N- and O-glycosylation. Glutaminyl and asparaginyl residues are frequently post-translationally deamidated to the corresponding glutamyl and aspartyl residues. Alternatively, these residues may be deamidated under mildly acidic conditions. Post-translational modifications include hydroxylation of proline and lysine, phosphorylation of hydroxyl groups of seryl or threonyl residues, methylation of the o-amino groups of lysine, arginine, and histidine side chains (acetylation of the N-terminal amine and, in some instances, amidation of the C-terminal carboxyl). Variations in post-translational modifications can include the variations in the type or amount of moieties posttranslationally attached to a proteins.

The terms “mutant,” “mutation” or “mutated sequence,” when used in reference to a polypeptide, protein, or nucleotide or amino acid sequence variations can be used interchangeably with the terms “variant,” “allelic variant,” “variance,” or “polymorphism.” Amino acid sequence modifications (“mutations”) include substitutions, insertions or deletions. Variations in an amino acid sequence may arise naturally as allelic variations (such as due to genetic polymorphism) or may be produced by human intervention (such as by mutagenesis of cloned DNA sequences), the examples being induced point, deletion, insertion and substitution mutants. Variations in a nucleic acid sequence may result in changes in the amino acid sequence, provide silent mutations, modify a restriction site, or provide other specific mutations. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence.

The term “lysis” and the related terms are used herein to refers to the breaking down of the membrane of a cell. A fluid or a suspension containing the contents of lysed cells is called a lysate.

The tern “solubilization” is used herein to denote the process of breaking interactions involved in protein aggregation, which include disulfide bonds, hydrogen bonds, van der Waals forces, ionic interactions, and hydrophobic interactions. Solubilization can be used in different contexts, for example, in the context of protein purification or disruption of protein-protein interaction. Some proteins, especially membrane proteins, may require detergents for solubilization. Detergents are classified as nonionic, zwitterionic, anionic, and cationic, and they may be used to disrupt hydrophobic interactions between and within proteins, as well as for other reasons. Some examples of the detergents that may be used for solubilization include sodium dodecyl sulfate (SDS; 0.1-10%), sodium deoxycholate (0.1-10%), Triton-X100 (0.01-5%), tween 20 (polysorbate 20; 0.01%-10%), Tween 80 (0.01-10%), CHAPS (0.01%-10%) and NP-40 (nonyl phenoxypoltethoxylethanol; 0.01%-10%). Elevated temperature may also be employed to achieve solubilization. For example, for some detergents, such as SDS, the temperature can be elevated to 50-99° C. for 1 min-18 hours. An incubation for 12 hours at 65° C. is commonly used for fixed samples. In another example, incubation at 99° C. for 1 hour may be used. Various denaturing agents, such as urea, may also be used.

The term “fixation” and the related terms are used herein to refer to processes used to stabilize proteins, nucleic acids and other components of cell and tissue samples. Fixation processes may involve exposure to chemical reagents referred to as “fixatives,” which typically make cell and tissue components, including proteins, insoluble. For examples, crosslinking fixatives, such as aldehydes (for example, formaldehyde and acrolein (propenal)) can create chemical bonds between proteins in a sample that anchor proteins to cytoskeleton. Effects of some cross-linking fixatives can be reversed. For example, formaldehyde crosslinking can be reversed by heating formaldehyde cross-linked sample in the presence of a salt, such as NaCl. Some other fixatives act by reducing the solubility of protein molecules and/or disrupting hydrophobic interactions. Some examples of fixatives that act as precipitating fixatives are alcohols, such as ethanol and methanol. Another example of a fixative is acetone. The samples may be exposed to a fixative (“fixed”) by various procedures. For example, a fixative may be injected in an animal and spread through the animal's body via blood flow in a process commonly called “perfusion.” The samples then may be prepared from the perfused animal's tissues. In another example, a cell sample may be immersed in a fixative and incubated in order to allow the fixative to diffuse. The duration of the procedure is determined by tissue type, size and density, as well as on the type of the fixative employed.

The term “permeabilization” is used herein to refer to processes used to make tissues, cell membranes or cell walls permeable to antibodies. Permeabilization may be achieved by exposure to a sample, to organic solvents and/or detergents. Some examples of detergents useful for permeabilization of the samples in the methods of the present invention are Triton X-100, Polyoxyethylene (20), sorbitan monooleate (Tween-20) or saponin.

### Detection Methods

The embodiments of the present invention include methods of identifying analyte molecules in proximity to a target molecule in a sample, which can also be referred to as “detection methods” and “methods of detecting.” The sample is a fixed, and optionally permeabilized, sample of a cell or a tissue. Fixation of the sample can be accomplished by any reversible cross-linking fixative reagent, including, but not limited to, formaldehyde or acrolein. Alternatively, fixation can be accomplished by precipitating fixatives, which can also be referred to as coagulants including, but not limited to, ethanol, methanol or acetone.

Prior to performing the methods of the present invention, a fixed sample may be permeabilized to facilitate diffusion and binding of the other reagents used in the methods of the present invention. Permeabilization may be accomplished by an exposure to a detergent including, but not limited to, Triton X-100, Polyoxyethylene (20) sorbitan monooleate (Tween-20) or saponin.

A sample may be a tissue sample having a thickness of up to several millimeters, for example, about 0.01-5 mm, 0.02-5 mm, 0.03-5 mm, 0.04-5 mm, 0.05-5 mm, 0.06-5 mm, 0.07-5 mm, 0.08-5 mm, 0.09-5 mm, 0.1-5 mm, 0.2-5 mm, 0.3-5 mm, 0.4-5 mm, 0.5-5 mm, 0.6-5 mm, 0.7-5 mm, 0.8-5 mm, 0.9-5 mm, 1-5 mm, 1.5-5 mm, 2-5 mm, 2.5-5 mm, 3-5 mm, 3.5-5 mm, 4-5 mm, or 4.5-5 mm. In comparison, a typical thickness of a cryo-sectioned unfixed sample is <100 μm.

The detection methods include a step of binding the primary binding molecule to the target molecule in the sample under the conditions that allow the binding of the target molecule in the sample to the primary binding molecule to occur. The conditions under which the binding occurs depend on the context of the specific method. For example, the binding may occur in a buffer with a suitable pH containing a mild detergent (such as Tween 20) and a common protein (such as bovine serum albumin (BSA)) for blocking non-specific interactions. Peroxide may be added to deactivate the peroxidases endogenous to the sample. Using an antibody as a primary binding molecule is envisioned, but other types of primary binding molecules may be used, as discussed elsewhere in this document.

Selection of the primary binding molecule, such as a primary antibody, is made based on the target molecule. The primary binding molecule is selected so that that it is specific for the target protein. Various degrees of specificity may be used, depending on the goals and the applications of the detection method. For example, in some instances, a primary binding molecule may be selected that specifically binds all or a larger subset of variants of a target molecule. In other instances, a primary binding molecule, such as a primary antibody, may be selected that specifically binds a smaller subset of variants, or only a particular variant. For example, in some embodiments, a primary antibody can be selected that specifically binds several variants and isoforms of a target protein. In some other embodiments of the present method a primary antibody is selected that is specific for the mutant protein target. In yet some other embodiments, the primary antibody is selected that is specific for the posttranslationally modified protein variant target. In still some other embodiments, the primary antibody is selected so that it is specific for a splice isoform. It is to be understood that primary binding molecules are not limited to antibodies and that, in some embodiments, a non-antibody protein or a non-protein affinity molecule may be used as a primary binding molecule.

A target molecule can be a protein. Such target protein may be a nuclear or a cytoplasmic protein, an extracellular or a secreted protein. Biological molecules that are not proteins can also be target molecules, including nucleic acids, lipids, fatty acids and carbohydrates, such oligosaccharides. A target molecule can be a part or a fragment of a biological molecule. For example, a target molecule may be a polypeptide, a nucleic acid sequence within a larger nucleic acid, an oligosaccharide within a posttranslational modified protein, etc. A target molecule may also be a nonbiological molecule, such as a small molecule, for example, a drug or a small-molecule ligand.

A target protein may be a nuclear envelope protein. Examples of target nuclear proteins are nuclear lamina proteins, such as lamin, nuclear pore proteins, and histones. Lamins include B type lamins (B1 and B2) and A-type lamins (A and C), which are splice variants of the product of the LMNA gene, as well as prelamin A/C precursor. Accordingly, a primary antibody may be selected so that it is specific for either lamin A or C, or so that it binds both variants. A histone target modification target may be, but is not limited to, acetylation, phosphorylation and different levels of methylation of H3K4, H3K9, H3K14, H3K27, H3K79, H3K36, H4K20 or H2BK5. A primary antibody may be selected so it binds to a particular histone or to particular posttranslationally modified variants of a histones. Some other exemplary target proteins are CTCF, P53, P53BP1, ATM, CHK1, CHK2, ATR, PRKDC, CAK, TP53RK, RAS, an mTOR complex protein, WRN, Ku70, Ku80, SREBP, APOE, LAP2 or a NF-κB complex protein.

In some embodiments of the methods described herein, after the binding of the primary antibody to the target protein in the sample, a secondary antibody is added to the sample and incubated under the conditions allowing the binding of the secondary antibody to the primary binding molecule in the sample to occur. In such embodiments, the secondary antibody is typically conjugated to peroxidase, although other ways of incorporating the peroxidase activity into the secondary antibody may be employed. In some other embodiments, the primary binding molecule is conjugated to peroxidase, or otherwise incorporates the peroxidase activity, and the secondary antibody is not employed. One example of a peroxidase that can be suitably conjugated to a secondary antibody or to a primary binding molecule is horseradish peroxidase. Another example of a peroxidase is APEX.

In yet some other embodiments of the detection methods described herein, a primary binding molecule may not be employed. For some target molecules, it may be hard to identify or generate a primary binding molecule, or binding of the primary molecule may interfere with other interactions. In that case, a peroxidase (such as HRP) may be added to the target molecule (for example, by click chemistry) and the peroxidase-conjugated molecule is incorporated into a cell or a tissue.

Subsequently to the above-described steps, biotin tyramide is added to the sample, and incubated with the sample under suitable conditions. In some cases, a solution of biotin tyramide in a reaction buffer is added to the sample. In some other cases, a solution of biotin tyramide is added to the sample prior to the addition of the reaction buffer and allowed to incubate with the sample prior to the addition to the reaction buffer, for example for 1 min-1 hour, to facilitate penetration of tyramide molecules into the sample. The reaction is allowed to proceed for 10 seconds-7 minutes, then stopped by addition of a reaction blocking reagent, such as sodium ascorbate. The reaction can be performed at room temperature, or at lower temperatures to reduce the rate of the reaction. The reaction buffer contains hydrogen peroxide, or other suitable peroxide molecules. The viscosity of the reaction buffer may also be altered by addition of various reagents, some of which are discussed below. Short-lived intermediates are generated, resulting in biotinylation of moieties in the proximity of the peroxidase. For example, Tyr, Trp, His and/or Cis residues in the proteins in the proximity of the peroxidase may be biotinylated. In another example, nucleic acids in proximity to the peroxidase may be biotinylated. In other words, biotinylation of one or more moieties in the molecules located in proximity to the peroxidase occurs.

In the context of the methods described herein, labelling proximity, which may be also called “labelling radius” may defined as a distance from the peroxidase and may be in the range of 20-200 nm. Labelling proximity may be influenced by various conditions, such as the biotinylation reaction time, components of the reaction buffer, and reaction conditions, such as temperature and viscosity. All the conditions may be used to change the labelling proximity and/or the scope of the molecules being biotinylated. For example, the biotinylation reaction time may be as short as 10 seconds or as long as seven minutes, with the increased reaction time resulting in greater labelling proximity. Biotinylation reaction temperature may be as high as 37° C. or (37° C. or less, for example, 0-37° C., or as low as peroxidase activity permits, as long as freezing is avoided—including sub-zero temperatures, depending on the buffer selected), with the higher temperature resulting in greater labelling proximity. Some of the reagents that may be incorporated into the reaction buffer to influence labelling proximity are viscosity altering reagents, such as glycerol, and bovine serum albumin (BSA), free radical scavengers, such as trolox, and sodium ascorbate.

It is to be understood that various blocking and washing procedures may be employed before or after any of the above methods steps. In other words, the methods of the present invention may include one or more washing and/or one or more blocking steps. Washing and blocking steps are employed, among other things, to decrease nonspecific binding and improve signal to noise ratios of the detection methods of the present intention. The reagents and the conditions selected for such steps may vary, but can be experimentally determined according to commonly known procedures. Some reagents that can be suitably incorporated into washing and blocking solutions are bovine serum albumin (BSA) and detergents. For example, PBS with or without 0.1% Tween, may be used as a washing reagent, as well as water. Deactivation of endogenous peroxidase is accomplished by adding hydrogen peroxide to the sample at a suitably high concentration (such as 0.5%) and incubation for 10 min.

To validate proper deposition of biotin, visualization or other types of the detection of the molecules containing biotinylated moieties may be performed by using avidin, streptavidin or related molecules with affinity to biotin and containing appropriate labels. For example, in some of the embodiments of the methods of the present invention, biotinylated proteins or other molecules in the samples may be visually detected, such as in slides examined or imaged under the microscope, using fluorescently labeled avidin or streptavidin. Fluorescence emitted by the label may be quantitatively detected by registering light emitted by the sample at a particular wavelength. The labels employed in the detection of biotinylated molecules are not limited to fluorescent labels. For example, for various types of detection, metals, spin-labeled molecules, nanoparticles, enzymes or radioisotopes may be used.

In some embodiments, after biotinylation, the sample may be treated under the conditions allowing for lysis of the cells, solubilization of the proteins and/or other components in the sample and/or reversal of cross-linking, either naturally existing, such as the cross-linking of lamin molecules in the lamina, or introduced by a reversible cross-linking fixative, if one was employed to produce the sample. For example if the sample was subjected to treatment with the reversible cross-linking reagent formaldehyde, then the reversal of the cross-linking can be accomplished by incubation at 55-99° C., optionally in the presence of NaCl or other salts. It is to be understood that the exposure of the sample to cross-linking reversal conditions may allow for partial reversal of the effects of the reversible cross-linking reagent. In another example, the sample may be subjected to elevated temperatures (for example, 55-99° C.), exposure to a detergent, such as SDS or sodium deoxycholate, exposure to a denaturing agent, such as urea, shaking, sonication (for tissue samples), and to other procedures devised to solubilize the sample components. It is to be understood that solubilization can be a partial solubilization. Upon solubilization, the sample may be centrifuged to separate the soluble fraction as a supernatant. or other suitable separation techniques may be employed, including size selection by filtration or dialysis.

Biotinylated proteins or polypeptides can be purified or isolated from the sample by a process comprising an affinity interaction. For example, avidin- or streptavidin-coated beads may be used to isolate a fraction containing biotinylated proteins and/or polypeptides by incubation of sample or its fraction (for example, of the soluble fraction) with the beads under the condition allowing for binding of biotinylated proteins or polypeptides to avidin or streptavidin moiety on the beads, followed by one or more washing steps to reduce or eliminate the non-specifically bound components. The biotinylated proteins or polypeptides can be released from the beads by the exposure to suitable conditions, such as a high-salt buffer, high temperature, changes in the pH or by other appropriate procedures. In another example, proteins may be digested directly on the beads, and the resulting peptides analyzed by mass spectrometry. The same or similar procedures may be applied to isolation of other analyte molecules, such as nucleic acids.

The isolated fraction containing biotinylated molecules, such as biotinylated proteins or polypeptides, may be then analyzed by various procedures and methods, including Western blot, mass spectrometry, sequencing, nucleic acid amplification or high throughput protein identification methods. One or more procedures and methods for analysis of proteins and other molecules may be selected and adapted to the methods described herein based on the application of the methods and the information desired. For example, calculation of the enrichment in the proteins found in the isolated fraction in relation to the proteins that remained unbound during affinity purification step (“ratiometric calculation”) may distinguish between the proteins in proximity to the target protein and the contaminations by the abundant proteins in the sample. The use of SILAC may detect even small changes in the composition and the abundance of the proteins in proximity to the target proteins under various conditions.

### Advantages and Uses

Detection methods described herein possess a number of advantages in comparison to other known proximity labelling methods. For example, unlike the method described in Hung et al., “Proteomic Mapping of the Human Mitochondrial Intermembrane Space in Live Cells via Ratiometric APEX Tagging,” Molecular Cell 55, 332-42, 2014, embodiments of the present detection methods can use the antibodies to bind a target molecule, such as a protein target, and to biotinylate the analyte molecules, such as proteins or nucleic acids, in close proximity. This allows for testing of any cell or tissue sample and does not require creation of genetically engineered peroxidase or creation of transgenic cells or animals expressing such genetically engineered peroxidase, employed in Hung et al. In the methods of the present invention, off-the-shelf reagents, such as commercially available primary and secondary antibodies can be used, respectively, for binding the target molecule and for biotinylation. The methods of the present invention are therefore easy to use and versatile. For example, the antibodies specific to known target protein variants, such as mutants or posttranslationally modified variants can be used in the methods described herein, and the changes in protein-protein and other molecular interactions associated with such variants may be detected.

Rees et al. “Selective Proteomic Proximity Labeling assay Using Tyramide (SPPLAT): A Quantitative Method for the Proteomic Analysis of Localized Membrane-Bound Protein Clusters” Current Protocols in Protein Science, 19.27.1-19.27.18, April 2015 and Li et al. “New Insights into the DT40 B Cell Receptor Cluster Using a Proteomic Proximity Labeling Assay” Journal of Biological Chemistry 289:14434-14447, 2014, describe the methods for detecting protein-protein interactions in membrane microenvironments that use a primary antibody and biotin tyramide labelling. However, the detection of proteins interacting with nuclear or cytoplasmic proteins is not described or suggested. The detection of PPIs in fixed and optionally permeabilized samples is also not described or suggested. The methods of the present invention allow for the detection of analyte molecules in fixed samples, thus permitting the testing for the samples previously obtained, such as clinical tissue sections or biopsy samples. Thus, the methods of the present invention overcome the limitations on the types of the samples that may be tested.

The methods of the present invention may be performed on permeabilized samples, which allows for penetration of the method reagents in the interior of the cells and detection of the interactions of difficult to reach targets, such as nuclear and cytoplasmic protein targets. Thus, the embodiments of the present invention overcome the limitation on the types of the target molecules and the interactions being detected. Furthermore, the methods of the present invention may be performed on tissue samples that are thicker than the cryo-sliced samples, such as those used in the above articles. The methods of the present invention can be performed on the tissue samples of the thickness up to 100-fold greater than the cryo-sliced samples disclosed in the articles. Thus, the methods of the present invention overcome the limitations on the types of the samples that may be used.

Furthermore, it was found that the reversal of the cross-linking, solubilization and other procedures employed in some embodiments of the detection methods described herein, such as solubilization of the samples under denaturing conditions, allows for disruption of the bonds created by the cross-linking reagents during fixation or naturally occurring in cells or tissues. This disruption allows for effective isolation and identification of the interacting molecules from the strongly associated complexes and other higher order structures, such as nuclear lamina. Such complexes and high order structures, if left un-dissociated, lead to isolation of multiple unrelated molecules, which masks the signal from the interactors of the target molecules and hinders analyte molecule identification. Embodiments of the methods of the present invention provide a solution to this problem.

It was also unexpectedly found that using, separately or in various combinations, various washing steps and reagents and other conditions, such as biotinylation reaction time and BSA blocking, in the methods according to the embodiments of the present invention, allows the ability to control the labelling proximity, which improves signal to noise ratios and specificity of detection of the interacting molecules. For example, embodiments of the methods of the present invention allow for isolation of protein fractions that contain substantial amounts of or almost exclusively the proteins found in very close physical proximity to the primary binding molecule. Accordingly, embodiments of the methods of the present invention provide solutions to the low signal to noise ratios and low specificity problems of some proximity labeling methods.

Various applications of the methods of the present invention are envisioned. In one application, a detection method employing mass-spectrometry analysis of biotinylated proteins successfully identifies multiple interactions of the nuclear envelope protein Lamin A/C, mutant versions of which cause Hutchinson-Gilford progeria syndrome (HGPS) and other laminopathies. The method of the present invention outperformed previously known methods of identifying lamin interactors. Accordingly, the methods of the present invention can be employed for identification of lamin interactors in various samples, for example, those obtained from different tissues.

The methods according to the present invention can be used to promote the understanding of PPIs and of the mechanisms by which the PPIs and variant protein interactors contribute to disease. In some examples, the methods of the present invention can be used for comparative identification of target proteins interactors from young and old subjects, healthy and sick subjects, or from animal model subjects, such as mice expressing progerin. Among other things, the methods of the present invention can be used to determine tissue specific interactors of target proteins, and to differentiate tissue vs. cell line interactors of the target protein.

The method according to the embodiments of the present invention may be applied to differential proteomics. Used in conjunction with SILAC, an embodiment of the method of the present invention reliably detects changes in lamin A/C protein-protein interactions in cells that are heat shocked. The methods of the present invention can therefore be used to detect changes in the levels and patterns of proteins interacting with a target protein when the cells or tissues are exposed to changing conditions, such as extreme temperatures, drugs, infection, toxicity, disease, etc. Application of the methods of the present invention to research uses, toxicity screening, forensics and pathology is envisioned. The methods of the present invention may be useful in a wide range of analytical, diagnostic, clinical and therapeutic applications, for example, in research and laboratory applications in which detection of protein-protein interactions is desirable, or in methods of assessing various conditions, such as laminopathies, in subjects.

### Diagnostic, Clinical and Therapeutic Methods

The detection methods described herein may be used in diagnostic as well as in the therapeutic methods, or in methods related to treating diseases or conditions. Some of these methods may be referred to as “screening” and/or “profiling” methods and may be employed in the fields of diagnostics, therapy, personalized medicine and other related fields.

The detection methods described herein may be used in various other methods, which can be characterized, depending on the context, as screening, diagnostic, therapeutic or treatment methods. In one example, the detection methods of the present invention may be employed in methods of detecting the presence, absence, pattern or amount of one or more target molecule interactors in a sample, where the presence, absence, pattern or amount of the one or more target molecule interactors, or change in one or more of those, detected in the sample with the help of the detection methods of the present invention, is indicative of the presence, absence or amount of exposure to a factor, such as temperatures, drugs, infection, toxicity or a disease or condition. In another example, the detection methods of the present invention may be employed in methods for detecting the presence, absence, pattern or amount of one or more target molecule interactors in a sample, where the presence, absence, pattern or amount of the one or more target molecule interactors, or change in one or more of those, detected in the sample with the help of the detection methods of the present invention, is indicative of the presence, absence or amount of a disease or a condition. In such situations, the presence, absence, pattern or amount of the one or more target molecule interactors in the sample may serve as a diagnostic marker (biomarker) of exposure to a factor, such as temperatures, drugs, infection, toxicity or a disease or conditions, or a diagnostic marker (biomarker) of the disease or the condition. Methods of diagnosing or monitoring a disease or a condition are also included among the embodiments of the present invention. For example, the detection methods of the present invention may be employed in methods of detecting a change in the presence, absence, pattern or amount of one or more target molecule interactors in a sample, over time or in comparison to a baseline standard, wherein the change is indicative of the onset, progression or development, or lack thereof, of a disease or a condition.

Methods of treating a disease or a condition in a subject that include administering a treatment or therapy in the subject based on the detection of the presence, absence, pattern or amount, or a change in one or more thereof, of the one or more target molecule interactors are also included among the embodiments of the present invention. The term “therapy” is used herein synonymously with the term “treatment,” and may include surgical treatments. A therapy may include one or more types of therapy. The detection methods described herein may be used in methods of determining efficacy of a therapy or a treatment in a subject based on a change or changes in the assessed interactions of a target molecule. Such methods can be described as pharmacodynamic methods, or methods of evaluating efficacy of a treatment or therapy. An exemplary method includes obtaining a first sample from a subject with a disease prior to treatment, assessing interactions of a target molecule (i.e., as a baseline measurement) in the first sample, obtaining a second sample from a subject after at least one treatment, assessing interactions of a target molecule in the second (i.e., as a means of assessing the treatment effect), and comparing the results of the first assessment and the second assessment. A change in the assessed interactions of the target molecule may indicate that the therapy is insufficiently effective and that a second therapy, an increase in dosing regimen (increased dosage or frequency using the current treatment agent), or a change in the therapy for the subject should be selected. Proper dosages and treatment methods, or changes in treatment regimens can be determined by accepted methods.

### Kits

Kits for performing the methods of the present invention are included among its embodiments. A kit is a set of components, comprising at least some components for performing the methods according to the embodiments of the present invention. Such a kit may or may not contain a primary binding molecule, such as a primary antibody, which may be otherwise available for the target molecule. A kit may include one or more of fixation reagents, permeabilizing reagents, biotinylated tyramide, HRP-conjugated secondary antibody, hydrogen peroxide in an appropriate buffer, FITC-avidin for staining validation, and an optimized solubilization buffer.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### Example 1

### Experimental Procedures

### Cells

Various cell lines were grown in MEM medium supplemented with 10% fetal bovine serum (FBS). For SILAC experiments, cells were grown in MEM medium lacking lysine and arginine, and supplemented with isotope-labelled lysine and arginine (“heavy” and “light”), as well as “light” isotope-labelled proline. Dialyzed fetal bovine serum (FBS) was used. The cell samples were fixed and permeabilized by incubation with 4% formaldehyde for 10 minutes, followed by incubation for 7 minutes with 0.5% Triton-X100 in PBS, and incubation for 10 minutes with 0.5% H₂O₂ (to deactivate endogenous peroxidases).

### Tissue Samples

Frozen human tissues samples were sliced into thin sections (0.1-3 mm) without thawing, using an aluminum stand on dry ice. For fixation (cross-linking), tissues slices were immediately placed into 4% formaldehyde in PBST (phosphate buffered saline (PBS) with 0.1% Tween 20) and incubated for 10 minutes. Mice were perfused with 4% formaldehyde, and the samples obtained from the perfused mice were cross-linked by incubation with 4% formaldehyde. After washing with PBST, the tissue samples were permeabilized by incubation in PBS with 0.5% Triton X-100 for 7 minutes, following by washing and incubation for 10 minutes in 0.5% H₂02. After washing, tissues were blocked with 1% bovine serum albumin (BSA) in PBST for 2 hours.

### Proximity Labelling

Cell or tissue samples were prepared as described above and incubated overnight in PBST with 1% BSA and a primary antibody (the concentration was antibody dependent, typically 1:200-1:1000 dilution of MAB3211). Incubation with the primary antibody was followed by 3 washes over 1 hour, then by incubation with secondary HRP-conjugated antibody (AB6789, Abcam, 1:1000 dilution, in PBST with 1% BSA) for 2 hours. Incubation with the secondary antibody was followed by 3 washes with PBST over 2 hours.

For tyramide signal amplification (TSA) reaction, antibody-labelled tissue samples were incubated with 3 μl of phenol-biotin solution (NEL700001KT, Perkin Elmer) in 150 μl reaction buffer (“amplification dilution”). In some cases, the samples were pre-incubated with phenol-biotin for 30 minutes, followed by the addition of the reaction buffer, to enable full penetration of the tyramide molecules into the sample.

TSA reaction was allowed to proceed for 30 seconds-7 minutes, and then stopped by the addition of 500 mM sodium ascorbate. The samples were then washed 3 times with PBST. In some instances, a fraction of the sample was removed at this point and tested for primary antibody deposition—by labeling the sample with Cy3-labelled anti-primary antibody, and for biotin deposition—by labeling with FITC-avidin. The labelled samples were visualized under the microscope.

### Analysis

Following proximity labelling, the analysis of the samples was performed. The samples were subjected to solubilization and affinity-based purification (“pull down”), then analyzed by various techniques. In an exemplary solubilization protocol of a formaldehyde-fixed sample, the volume of the sample was adjusted to 0.1 ml. 30 μl of 10% SDS and 20 μl of sodium 10% deoxycholate, w/w were added to the sample, and it was then incubated at 99° C. in a dry bath for 30 minutes with mild shaking, sonicated (tissue sample only), then incubated again for 1 hour at 99° C. The sample was then centrifuged at 13,000 RPM for 5 minutes.

For affinity-based purification, the volume of the supernatant obtained after the centrifugation was adjusted to 1 ml with PBST, and 250 μl of a suspension of pre-washed streptavidin-coated beads were added to the supernatant. Following the addition of the streptavidin-coated beads, the sample was incubated for 2 hours at room temperature, followed by one wash in PBST, two washes with PBST with 1M NaCl, and two more washes with PBS. The sample volume was then reduced to 100 μl by removing some of the liquid while keeping the beads with a magnet (if magnetic beads were employed), or by centrifugation.

In some studies, following the solubilization and the affinity purification, a portion of the sample (˜10%) was used for Western blots to validate candidate targets and overall protein labelling with biotin. The remaining portion of the sample (˜90%) was used for mass-spectrometry analysis.

### Example 2

### Lamin A/C Proximity Labeling in HeLa Cells and Human Tissue Samples

### Deposition of the Primary and Secondary Antibodies During Proximity Labeling

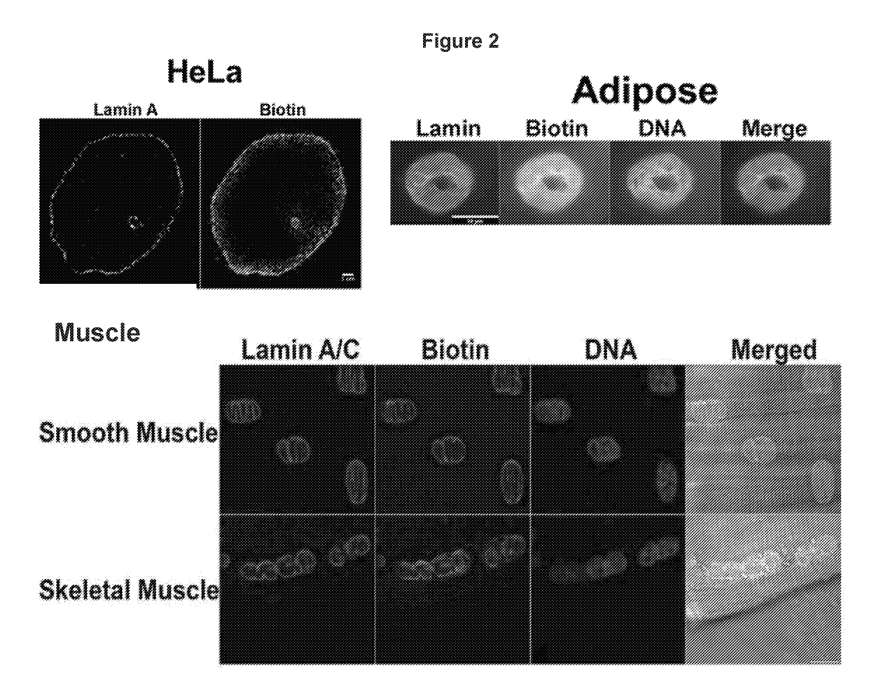

The samples of HeLa cells and of frozen post-mortem human adipose and muscle tissue were prepared and proximity-labelled, substantially as described in Example 1, using 3211 MAB (Merck Millipore) anti-lamin A/C antibody, recognizing both LMNA isoforms. The samples were tested for proper antibody staining by visualizing the primary antibody with Cy3 goat anti mouse antibody (AB97035, Abcam) and biotin deposition (labelled with FITC-avidin), also substantially as described in Example 1, and visualized. The results are illustrated in FIG. 2, which shows antibody staining and biotin deposition in the nuclear envelope. The proximity labelling revealed that nuclear envelopes of the human tissue samples deviated considerably from the classical view (smooth round sphere around the nucleus). For example, muscle fibers created grooves in the nucleus, which dictated not only the distribution of Lamin A/C, but also of DNA (FIG. 2, panel “Muscle”). In adipose tissue, some nuclei had a doughnut shape (FIG. 2, panel “Adipose”). This phenotype was not previously reported in primary human tissue samples.

### Identification of Lamin A/C Interactors by Proximity Labelling

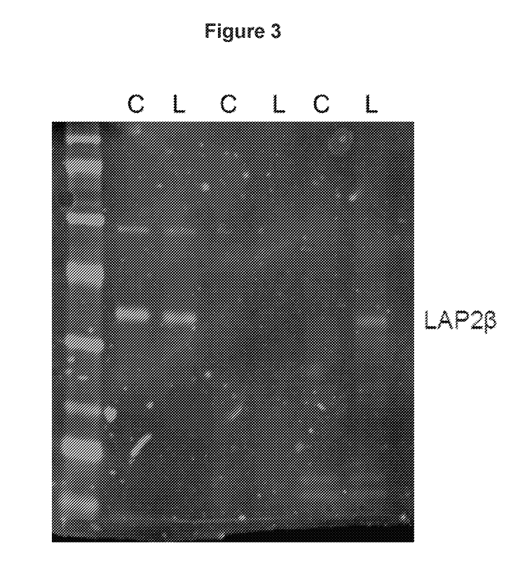

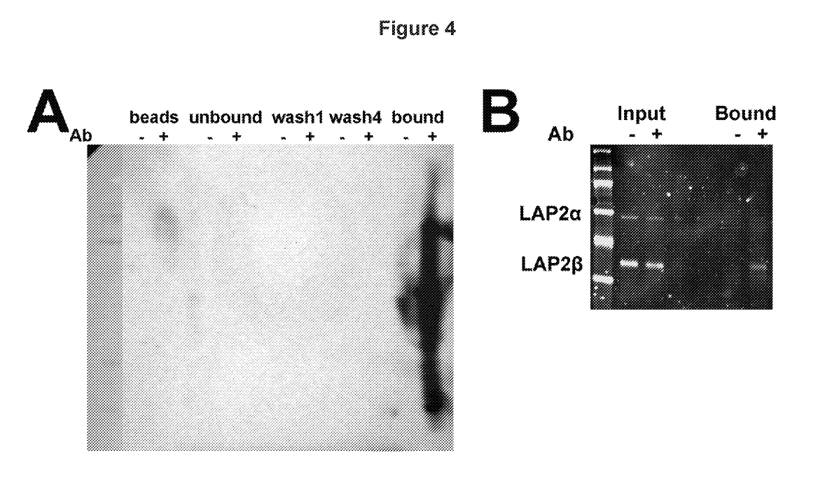

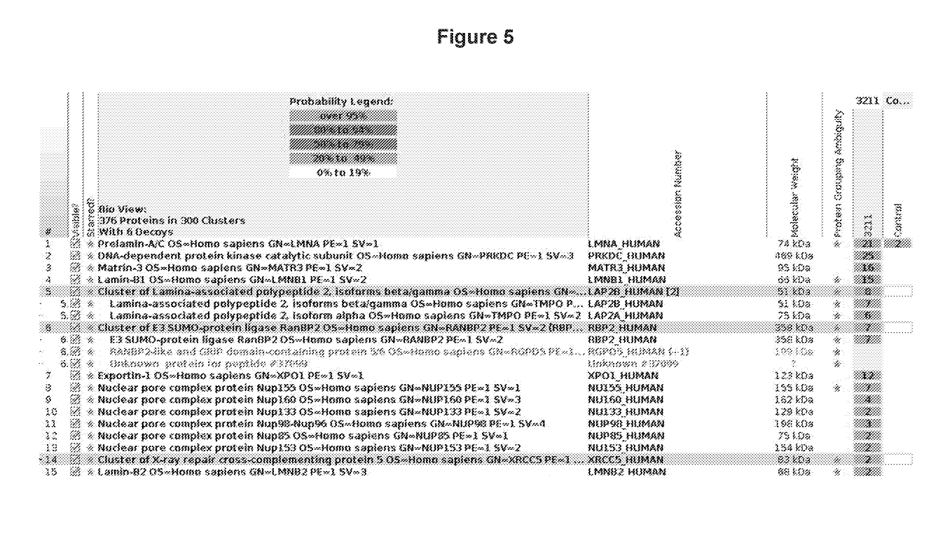

Identification of lamin A/C interactors was performed in the samples of HeLa cells and of frozen post-mortem human adipose and muscle tissue. Biotinylated proteins were pulled-down with streptavidin-coated beads and analysed by Western blot and mass-spectrometry. Some experiments conducted on HeLa cell samples revealed that LAP2β lamin interactor was isolated by affinity purification, as illustrated by FIGS. 3 and 4. In addition to identifying the targeted lamin A/C, multiple known interactors of lamin A/C were identified, including lamin B1, lamin B2 and lamina-associated polypeptide 2 (LAP2), as illustrated by FIG. 4, panel B and FIG. 5. These proteins were either found exclusively or enriched in the sample as compared to the control, as illustrated by FIG. 5.

Some proteins were found exclusively or with greater abundance in a particular sample. For example, nesprin proteins (SYNE1 and SYNE2), which are are known lamin A/C interactors and members of the Linker of Nucleoskeleton and Cytoskeleton (LINC) complex, were not detected in most of HeLa samples and ore were detected with a low peptide count of one to two peptides. In contrast, nesprins were identified in all the human muscle samples tested with a unique peptide count of up to 14. Similarly, Muscular LMNA-interacting protein (MLIP) was found in all the muscle tissue samples, but not in HeLa cell samples or adipose tissue samples.

Out 141 nuclear envelope proteins identified with two or more peptides in skeletal muscle tissue samples, 132 were also identified, without any filtering criteria, in smooth muscle tissue samples. By contrast, only 60 of these proteins were detected in adipose tissue samples. These results suggested that nuclear envelope composition varied significantly among different tissues. When compared with known disease causing proteins, skeletal muscle tissue showed nuclear envelope enrichment for several proteins mutated in muscular dystrophies and cardiomyopathies, including multiple members of the dystrophin-glycoprotein complex.

### Example 3

### Alpha-Sarcoglycan Proximity Labeling

Proximity labelling was used to identify proteins in close proximity to Alpha-sarcoglycan (SGCA), a member of the dystrophin-glycoprotein complex. By applying the method on human skeletal muscle tissue, using anti SGCA (Mouse Monoclonal IgG1 Clone #769109; R&D systems), multiple members of the dystrophin-glycoprotein complex were successfully identified. Theu included sarcoglycan units beta (SGCB), gamma (SGCG) and delta (SGCD), dytrophin (DMD), dystroglycan (DAG1) and sarcospan (SSPN).

### Example 4

### Specificity and Specificity of Proximity Labelling

“Ratiometric analysis” was used to improve the performance of proximity labelling. During proximity labelling, reaction time determined the labeling radius and signal intensity. While limiting the labeling radius reduced the number of non-nuclear envelope proteins identified, improving the specificity, it also decreased the signal intensity both for rare and abundant nuclear envelope proteins. In contrast, increasing the reaction time resulted in signal arising from leakage to non-nuclear envelope proteins. To improve the performance of proximity labelling, a ratiometric enrichment score was used, which compared the biotin-bound fraction with the unbound fraction. For proteins in proximity to the target, biotin labeling drove the enrichment in the bound over the unbound fraction. In contrast, as the signal from the decays with the distance from the target, abundant proteins that are labeled due to signal leakage were underrepresented in bound compared to the unbound fraction.

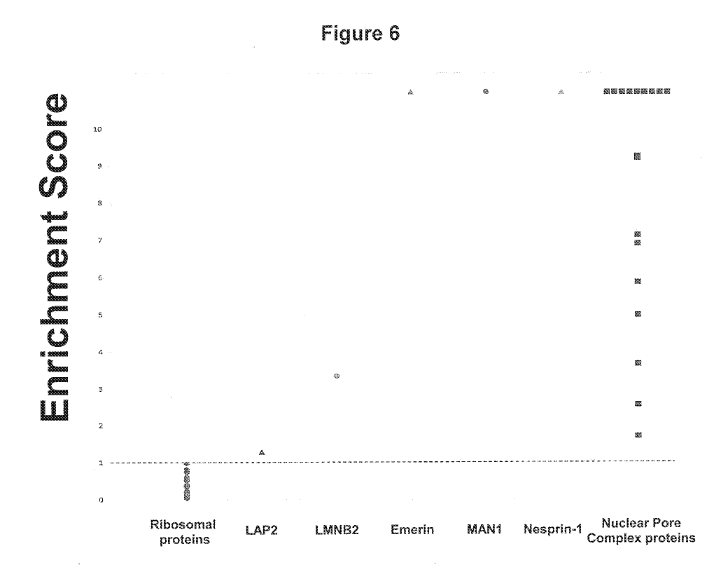

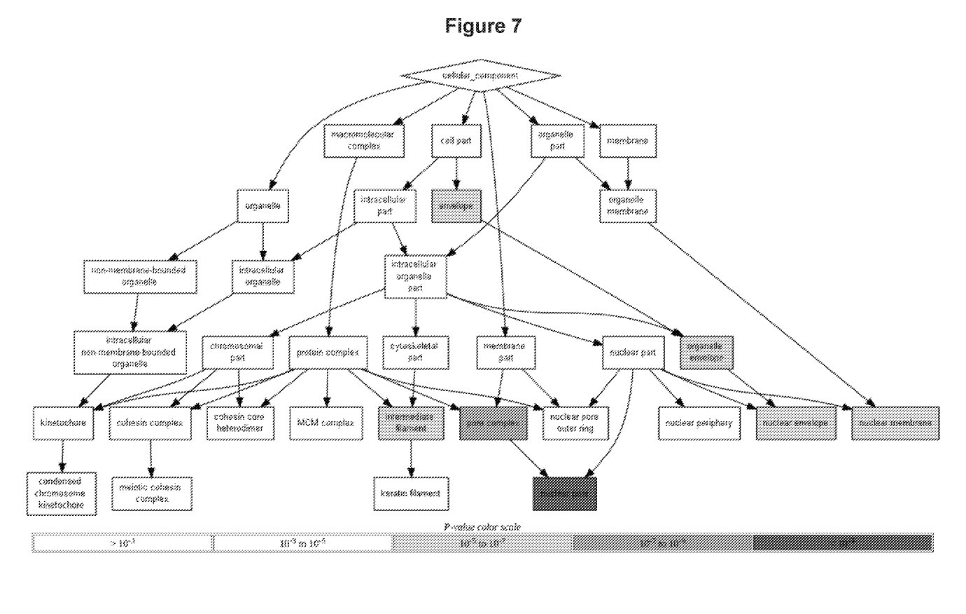

Mass-spectroscopy analysis of proximity-labelled HeLa cell samples revealed that the isolated fractions of the samples were significantly enriched (enrichment levels above 1) for nuclear pore complex proteins and other known lamin interactors, while low enrichment levels (below 1) were observed for abundant ribosomal proteins, which are common contaminants in nuclear envelope extracts. The results are illustrated by FIG. 6. A classification of the enriched proteins by origin (illustrated by FIG. 7) also revealed significantly higher enrichment levels for nuclear pore complex proteins, in comparison to the proteins of other origins. The above results demonstrated specificity of the labeling of the nuclear pore proteins in proximity to lamin.

TABLE 1
Detection of nuclear pore proteins.

| Gene Name | Aliases | Found |
|---|---|---|
| NUP188 | | Yes |
| NUP54 | | Yes |
| NUN43 | | Yes |
| NUM37 | | Yes |
| NUP35 | | Yes |
| SEH1L | SEH1 | Yes |
| NDC1 | | Yes |
| NUPL1 | Nup58 | Yes |
| NUP107 | | Yes |
| NUP133 | | Yes |
| NUP153 | | Yes |
| NUP155 | | Yes |
| NUP160 | | Yes |
| NUP205 | | Yes |
| NUP214 | | Yes |
| NUP85 | Nup75 | Yes |
| NUP88 | | Yes |
| NUP93 | | Yes |
| NUP98 | NUP96 | Yes |
| NUP62 | | Yes |
| NUP210 | | Yes |
| AAAS | Aladin | Yes |
| SEC13 | | Yes |
| RAE1 | | Yes |
| TPR | | Yes |
| RANBP2 | NUP358 | Yes |
| POM121 | | No |
| NUPL2 | NLP1 | No |
| NUP50 | | No |

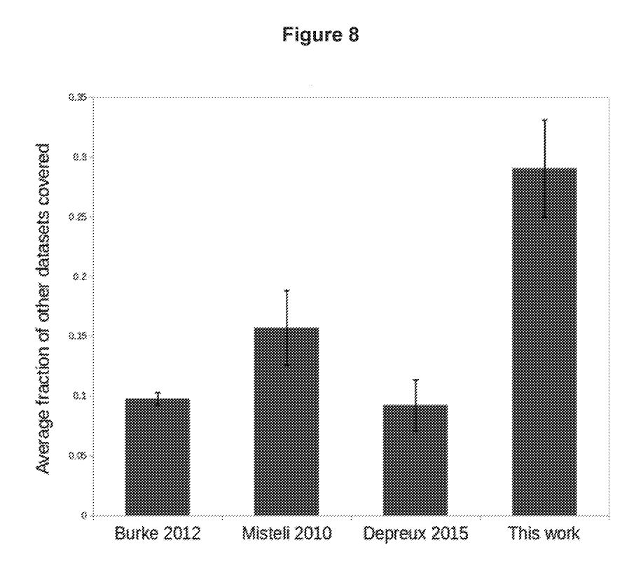

A comparison of three published lamin A-binding-protein datasets (Depreux et al. “Disruption of the lamin A and matrin-3 interaction by myopathic LMNA mutations” Human Molecular Genetics 24(15)4284-4295, 2015 (“Depreux”); Kubben et al. “Identification of differential protein interactors of lamin A and progerin” Nucleus 1(6):513-525, 2010 (“Misteli”) and Roux et al. “A promiscuous biotin ligase fusion protein identifies proximal and interacting proteins in mammalian cells” J. Cell. Biol. 196(6):801-810, 2012 (“Burke”)) with the list of the proteins identified according to the methods described in Example 1 revealed that the method described herein was significantly more sensitive in comparison to the previously known methods. The results of the comparison are illustrated by FIG. 8. For each dataset, it was calculated what percentage of the other datasets is covered by it.

Analysis of the proximity-labelled samples of HeLa cells was performed substantially as described in Example 1 and revealed that the method of the present invention identified 27 out of 30 nuclear pore proteins listed in Le Sage et al. “Viral subversion of the nuclear pore complex” Viruses 5(8):2019-2042. The nuclear pore proteins identified are listed in Table 1. Thus, sensitivity of the method described herein was demonstrated.

### Example 5

### Differential Proteomics

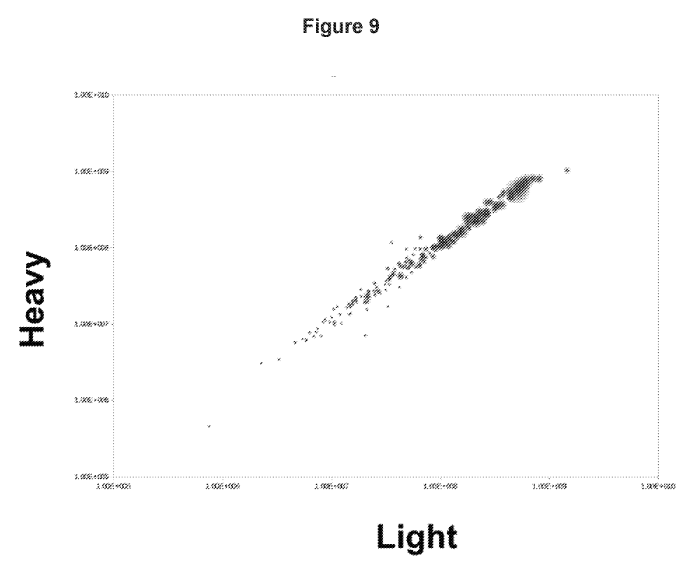

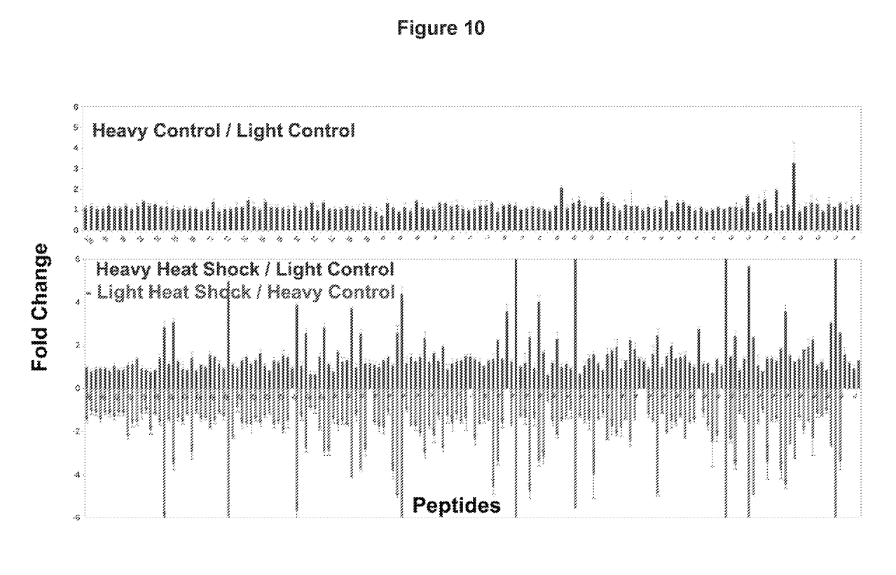

A study of HeLa cells was conducted using proximity labelling in conjunction with SILAC. HeLa cells were subjected to 43° C. heat shock for 2 hours, and the changes in the composition of the nuclear lamina were evaluated. Control HeLa cells were not subjected to the heat shock. “Light” cells were grown in culture with ¹²C6; ¹⁴N4-Arg and ¹²C6; ¹⁴N2-Lys. “Heavy” cells were grown in culture with ¹³C6; ¹⁵N4-Arg and ¹³C6; ¹⁵N2-Lys. The “light” and the “heavy” cells were mixed and subjected to proximity-based labelling. Peptide isolation was performed substantially as discussed in Example 1. The isolated peptides were analyzed by mass-spectrometry. FIG. 9 illustrates the reproducibility of the method employed. The observed correlation between nuclear envelope proteins of HeLa cells grown in heavy and light SILAC medium was R²=0.986. FIG. 10, which shows, in the upper panel the peptide profile illustrating comparison of the “heavy” vs. “light” control cells and, in lower panel, shows the peptide profiles of the “heavy” heat shocked cells vs. “light” control cells (top bar graph) and of the “light” heat shocked cells vs. “heavy” control cells (bottom bar graph). The number of peptides used to calculate the fold change in Y is plotted on the X axis. The Y axis shows fold change. The results showed significant changes in the pattern of lamin interactions occurring after the heat shock.

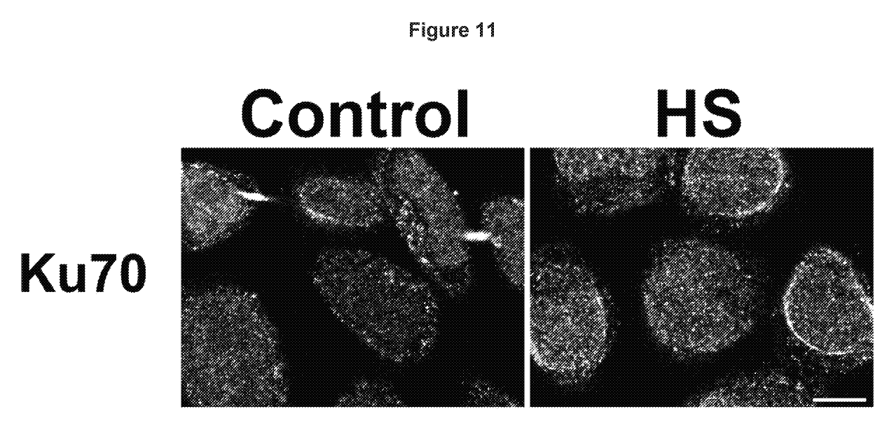

As expected, in the control experiments, illustrated by the top panel of FIG. 10, the “heavy” to “light” ratio was very close to one, particularly when multiple peptides were used to calculate the ratio. In contrast, when comparing a control with a heat shocked sample, multiple proteins displayed changed “heavy” to “light” ratios, as illustrated by the bottom panel of FIG. 10. The proteins identified among those displaying changed ratios included multiple heat shock proteins. Elevated levels of non heat-shock proteins were also detected in the heat-shocked cells. For example, elevated levels of Ku70 and Ku80 proteins (also known as XRCC6 and XRCC5, respectively), were are regulators of DNA-PKcs and members of the NHEJ pathway, were detected. Ku70 was previously identified as a lamin AC binding protein. Ku70 and Ku80 were found to be ˜2.5-fold more abundant in the nuclear envelope following heat shock (p=10⁻⁵ and 0.0005 for Ku70; p=0.007 and 0.002 for Ku80 for each of the two experiments). HSPA8, known to bind Ku70 and suppress its nuclear import, showed a similar increase (p<10⁻⁶ for each of the two experiments). By contrast, no change was seen for the levels of DNA-dependent protein kinase catalytic subunit (DNA-PKcs, also known as PRKDC). To confirm that the above changes reflected changes in molecular localization, and not protein abundance, we visualized Ku70 and Ku80 by imaging HeLa cells expressing green fluorescent protein (GFP)-Ku70 and GFP-Ku80 fusion proteins during heat shock. The total protein levels were not found to increase during a 2-hour heat shock. The results are illustrated in FIG. 11. The changes in Ku70 and Ku80 levels were also found to be heat-shock specific, as UV irradiated cells showed a mild decrease in nuclear envelope Ku70 and Ku80 abundance.

### Example 6

### Progerin Proximity Labeling in Transfected HeLa Cells

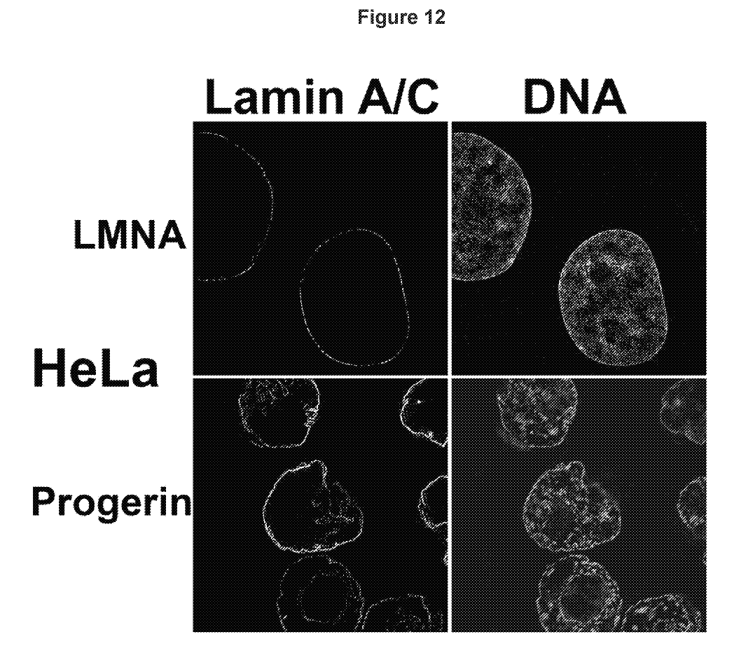

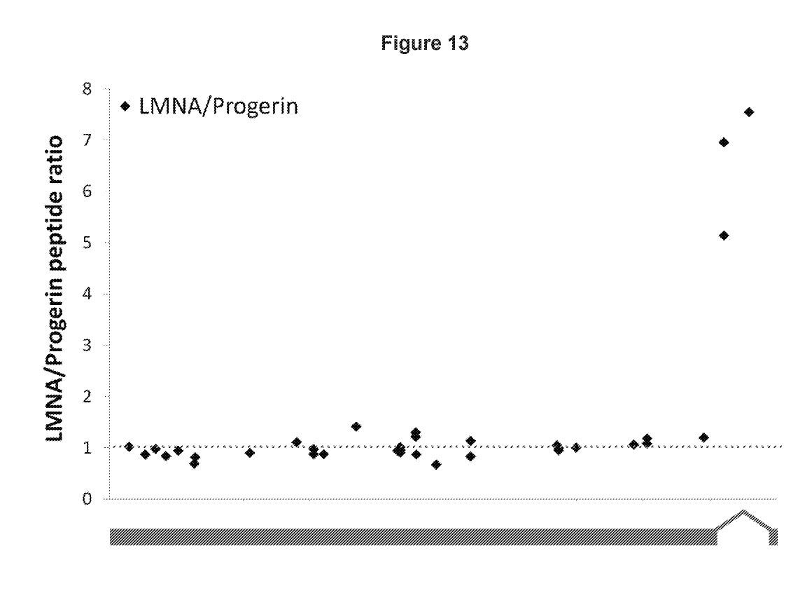

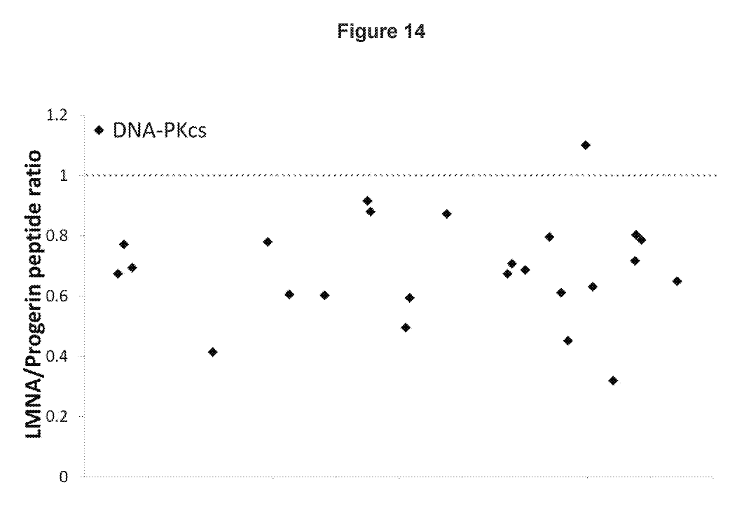

Proximity labelling in conjunction with SILAC was applied to HeLa cells transfected with GFP-LMNA or GFP-progerin. An anti GFP antibody was used to direct biotin labeling. By observing the cells 24 hours after transfection, the changes in the composition of the nuclear envelope resulting directly from the expression of progerin, rather than from later byproducts of senescence, were detected, as illustrated by FIG. 12. By observing the cells 24 hours after transfection, the changes to the composition of the nuclear envelopes resulting directly from the acute expression of progerin, rather than from later byproducts of chronic expression, such as cellular senescence, were detected. As expected, the heavy to light ratio for most lamin A/C peptides was close to one. Only three peptides exhibited a significant deviation from this ratio, all of which overlapped with the exon 11-encoded 50 amino acid stretch found only in lamin A/C and not in progerin, thus demonstrating our ability to distinguish between isoforms even in a wild-type lamin A/C endogenous background (FIG. 13). Among the proteins preferentially binding progerin was DNA-PKcs, previously reported to bind progerin (FIG. 14). Additionally, progerin was found to preferentially bind Ku70 and Ku80, for which progerin binding was not previously reported.

### Example 7

### Nuclear Envelope Proximity Labeling in Primary HGPS Fibroblasts

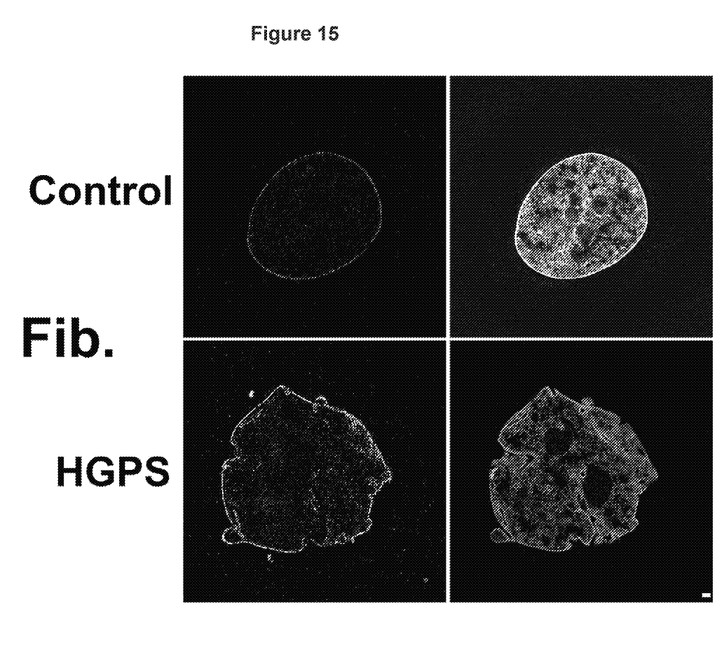

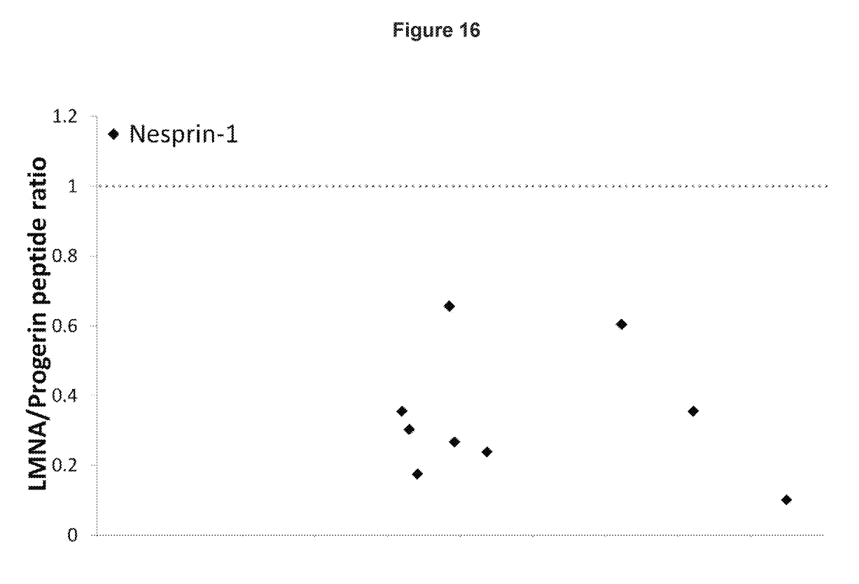

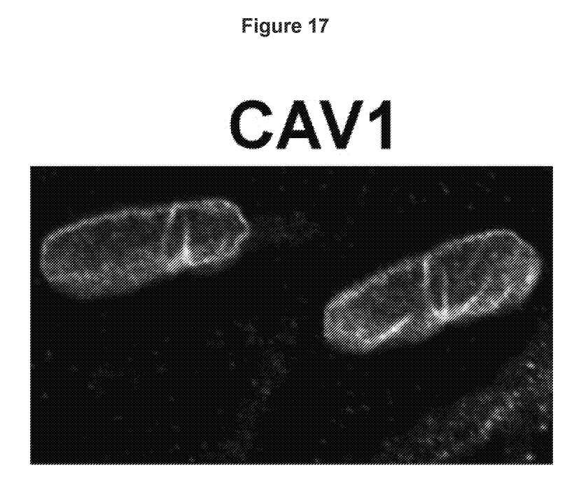

To detect the changes in in composition resulting from progerin expression, we grew primary control fibroblasts in heavy medium and compared the nuclear envelope composition with fibroblasts donated by a HGPS patient. MAB3211 was used. See FIG. 15. The changes in the abundance of several known nuclear envelope proteins, including Nesprin-1 (see FIG. 16), Sun2 and RanBP2 were detected. Additionally, in HGPS fibroblasts we saw a NE decrease in Polymerase I and transcript release factor (PTRF) and its interactor caveolin-1 (CAV1). Mutations in PTRF and Caveolin family member proteins are known to be associated with lipodystrophy and muscular dystrophy. To gain further insight into the roles of CAV1, it was immunostained on HGPS fibroblasts. While some CAV1 foci were seen at the nuclear periphery, only a small fraction of CAV1 localized near the nuclear membrane and may have been available for nuclear envelope interactions. In human tissue, clear nuclear envelope localization of CAV1 with a more smooth distribution was seen, suggesting a significant fraction of CAV1 is found at the nuclear membrane. See FIG. 17.

All patents, patent applications, publications, and abstracts cited above are incorporated herein by reference in their entirety. Various embodiments of the invention have been described in fulfillment of the various objectives of the invention. It should be recognized that these embodiments are merely illustrative of the principles of the present invention. Numerous modifications and adaptations thereof will be readily apparent to those of skill in the art without departing from the spirit and scope of the invention as defined in the following claims.

1. A method of identifying one or more analyte molecules in proximity to a target molecule in a sample, comprising:
contacting the sample with a primary binding molecule under primary molecule binding conditions, which allow binding of the target molecule in the sample to the primary binding molecule to occur, wherein the sample is a fixed and optionally permeabilized sample of a cell or a tissue;
subsequent to contacting, exposing the sample to a secondary antibody under secondary antibody binding conditions allowing binding of the secondary antibody to the primary binding molecule to occur in the sample, wherein the secondary antibody comprises a peroxidase;
subsequent to exposing, adding biotin tyramide to the sample under biotinylation conditions allowing for biotinylation of one or more moieties of the one or more analyte molecules located in proximity to the peroxidase, thereby resulting in one or more biotinylated moieties;
subsequent to adding, treating the sample under conditions allowing for protein solubilization;
subsequent to treating, isolating from the sample the one or more analyte molecules or one or more parts thereof comprising the one or more biotinylated moieties, by an isolation process comprising affinity interaction of the one or more biotinylated moieties and an affinity reagent; and,
identifying the one or more analyte molecules or the one or more parts thereof.

2. The method of claim 1, wherein the secondary antibody is conjugated to the peroxidase.

3. A method of identifying one or more analyte molecules in proximity to a target molecule in a sample, comprising:
contacting the sample with a primary binding molecule under primary molecule binding conditions, which allow binding of the target molecule in the sample to the primary binding molecule to occur, wherein the sample is a fixed and optionally permeabilized sample of a cell or a tissue, and wherein the primary binding molecule comprises a peroxidase;
subsequent to contacting, adding biotin tyramide to the sample under biotinylation conditions allowing for biotinylation of one or more moieties of the one or more analyte molecules located in proximity to the peroxidase, thereby resulting in one or more biotinylated moieties;
subsequent to adding, treating the sample under conditions allowing for protein solubilization;
subsequent to treating, isolating from the sample the one or more analyte molecules or one or more parts thereof comprising the one or more biotinylated moieties, by an isolation process comprising affinity interaction of the one or more biotinylated moieties and an affinity reagent; and,
identifying the one or more analyte molecules or the one or more parts thereof.

4. The method of claim 3, wherein the primary binding molecule is conjugated to the peroxidase.

5. (canceled)

6. The method of claim 1, wherein the target molecule is a protein, a polypeptide, a lipid, a fatty acids, an oligosaccharide, a nucleic acid, or a small molecule.

7-8. (canceled)

8. The method of claim 6, wherein the target molecule is a protein or a polypeptide.

9-10. (canceled)

11. The method of claim 1, wherein the one or more analyte molecules comprise a protein, a polypeptide, a lipid, a fatty acids, an oligosaccharides, a nucleic acid, a small molecule or a combination of two or more thereof.

12-13. (canceled)

14. The method of claim 1, wherein the primary binding molecule is a primary antibody.

15. (canceled)

16. The method of claim 1, wherein the fixed sample is a sample fixed by a reversible cross-linking reagent.

17. (canceled)

18. The method claim 16, wherein the treating step comprises exposing the sample to cross-linking reversal conditions allowing for at least partial reversal of effects of the reversible cross-linking reagent.

19-21. (canceled)

22. The method of claim 1, wherein the treating step comprises exposing the sample to a detergent, an elevated temperature, or both, under solubilization conditions allowing at least partial solubilization of proteins in the sample.

23. The method of claim 1, wherein the sample is permeabilized.

24. The method of claim 23, wherein the sample is permeabilized by exposure to a detergent.

25. The method of claim 24, wherein the detergent comprises one or more of Triton X-100, Polyoxyethylene (20), sorbitan monooleate (Tween-20) or saponin.

26. The method of claim 1, wherein the sample is a tissue sample having a thickness of 0.1-5 mm.

27. The method of claim 1, wherein, in the analyzing step, the one or more analyte molecules are analyzed a process comprising Western blotting, high throughput protein identification methods, nucleic acid amplification sequencing, mass spectrometry or a combination of two or more thereof.

28. The method of claim 1, wherein the affinity interaction is biotin-avidin or biotin-streptavidin interaction.

29. The method of claim 1, wherein the affinity reagent is a plurality of beads coated with avidin or streptavidin.

30-41. (canceled)

42. The method of claim 1, wherein the target molecule is a nuclear or a cytoplasmic protein.

43. The method of claim 42, wherein the target molecule is a nuclear envelope protein or a histone.

44-45. (canceled)

46. The method of claim 1, wherein the target molecule is a protein selected from the group consisting of CTCF, P53, P53BP1, ATM, CHK1, CHK2, ATR, PRKDC, CAK, TP53RK, RAS, an mTOR complex protein, WRN, Ku70, Ku80, SREBP, APOE, LAP2 and a NF-κB complex protein.

47. A method of identifying one or more analyte molecules in proximity to a target molecule in a sample of a cell or a tissue, comprising:
contacting the cell or the tissue with the target molecule under incorporation conditions allowing incorporation of the target molecule into the cell or the tissue, wherein the target molecule comprises a peroxidase;
after the incorporation, preparing the sample of the cell or the tissue, wherein the sample is a fixed and optionally permeabilized sample of the cell or the tissue, and;
adding biotin tyramide to the sample under biotinylation conditions allowing for biotinylation of one or more moieties of the one or more analyte molecules located in proximity to the target molecule, thereby resulting in one or more biotinylated moieties;
subsequent to adding, treating the sample under conditions allowing for protein solubilization;
subsequent to treating, isolating from the sample the one or more analyte molecules or one or more parts thereof comprising the one or more biotinylated moieties, by an isolation process comprising affinity interaction of the one or more biotinylated moieties and an affinity reagent; and,
identifying the one or more analyte molecules or the one or more parts thereof.

48. The method of claim 47, wherein the target molecule is a protein, a peptide, a small molecule, a drug, a nucleic acid, or a ligand.

49. The method of claim 47, wherein the peroxidase is conjugated to the target molecule.

50. A kit for performing identifying one or more analyte molecules in proximity to a target molecule in a sample, wherein the kit comprises a primary binding molecule that primarily binds to the target molecule, a secondary binding molecule that primarily binds to the primary binding molecule, a protein solubilization reagent and an affinity reagent.

51. (canceled)
